(19) United States
(12) Patent Application Publication
(10) Pub. No.: US 2011/0306827 A1
(43) Pub. Date: Dec. 15, 2011

(54) NEEDLE GUARD TO PROTECT ACCESS PORT TUBING

(75) Inventors: Kaustubh S. Chitre, Goleta, CA (US); Babak Honaryar, Orinda, CA (US); Ahmet Tezel, Goleta, CA (US); Nicholas J. Manesis, Summerland, CA (US)

(73) Assignee: ALLERGAN, INC., Irvine, CA (US)

(21) Appl. No.: 13/178,392

(22) Filed: Jul. 7, 2011

Related U.S. Application Data
(63) Continuation-in-part of application No. 13/105,715, filed on May 11, 2011.
Application No. 13/105,715 is a continuation-in-part of application No. 13/021,523, filed on Feb. 4, 2011.
(60) Provisional application No. 61/409,440, filed on Nov. 2, 2010.
(60) Provisional application No. 61/301,910, filed on Feb. 5, 2010.

(51) Int. Cl. A61F 2/02

(52) U.S. Cl. 600/37

(57) ABSTRACT

An inflatable tissue expander, suitable for implantation in a breast, is provided. In addition, a needle guard assembly, suitable to protect tubing leading from an implantable access port, is provided. The needle guard assembly may include a first composite guard and a second composite guard, each composite guard including an arrangement of puncture resistant members and a flexible substrate having a first side on which the puncture resistant members are positioned. The needle guard assembly may comprise a sleeve extending over an end of the tube.

### RELATED APPLICATIONS

This application is a continuation-in-part of U.S. patent application Ser. No. 13/105,715, entitled “INFLATABLE PROSTHESES AND METHODS OF MAKING SAME,” filed on May 11, 2011, which is a continuation-in-part of U.S. patent application Ser. No. 13/021,523, entitled “INFLATABLE PROSTHESIS AND METHODS OF MAKING SAME,” filed on Feb. 4, 2011, which claims the benefit and priority of U.S. Provisional Patent Application No. 61/301,910, filed on Feb. 5, 2010, and the benefit and priority of U.S. Provisional Patent Application No. 61/409,440, filed on Nov. 2, 2010, the entire disclosure of each of these applications are hereby incorporated by reference herein.

### FIELD

The present invention generally relates to medical implants and more specifically relates to inflatable prostheses, such as tissue expanders, suitable for implantation in a mammal, and also relates to tube protectors for use with implantable access ports.

### BACKGROUND

Prostheses or implants for reconstruction and/or augmentation of the human body are well known.

Fluid filled prostheses, for example, mammary prostheses or breast implants, are widely used to replace excised tissue, for example after a radical mastectomy, or to augment the body to improve surface configurations. Although there are many applications where these are used, the most common is the mammary prosthesis, used to augment or otherwise change the size or shape of the female breast.

A conventional saline-filled breast implant includes an outer shell of several layers of silicone elastomer having a valve or fill port. The prosthesis is typically implanted into the breast cavity in an empty or only partially filled state. The implant is then inflated to its final size by means of the valve or the fill port. This helps reduce the size of the needed incision, and enables a surgeon to adjust and even micro-adjust the volume of the implant. Unfortunately, the valve or the fill port is sometimes noticeable to the touch.

Many or even most implants are manufactured to a given size and shape, and are implanted without means or expectation of changing their size after implantation or initial filling when first inserted into the breast. However, in many situations it is desirable to be able to adjust the size of the implant over a substantial period of time. If the volume can later be adjusted, an implant of lesser initial volume can be implanted, and as the post-surgical swelling goes down, the implant used as a prosthesis can be enlarged. Also, because often the procedure is for cosmetic purposes, it is useful to be able to make a later adjustment of size without having to replace the prosthesis with one of a different size, which would require a subsequent surgical procedure.

One problem with many conventional adjustable implants is that they require a valve to be part of the implant.

It would be advantageous to provide an adjustable volume implant which does not require a valve or other access port for receiving fluid for adjustment.

Prior to implantation of a more permanent prosthesis, it is common practice to utilize a more temporary implant, for example, what is known as a “tissue expander” in order to gradually create the space necessary for the more permanent prosthesis. Keeping living tissues under tension by means of a tissue expander causes new cells to form and the amount of tissue to increase. Conventionally, a tissue expander comprises an inflatable body, having an inflation valve connected thereto. The valve may be formed into the inflatable body itself or may be remotely located and connected to the inflatable body by means of an elongated conduit.

The inflatable body of the tissue expander is placed subcutaneously in the patient, at the location of where tissue is to be expanded. The inflation valve, whether on the implant or remote thereto, is also subcutaneously positioned or implanted, and is configured to allow gradual introduction of fluid, typically saline, into the inflation body, by injection with a syringe. After gradual inflation at pre-determined intervals, the skin and subcutaneous tissues overlying the expander are consequently caused to expand in response to the pressure exerted upon such tissues by the inflatable body as solution is gradually introduced therein.

After gradual inflation at pre-determined intervals, which may extend over weeks or months, the skin and subcutaneous tissue will expand to the point where further medical procedures can be performed, such as the permanent implantation of a prosthesis, plastic and reconstructive surgery, or for use of the skin and subcutaneous tissue for use in some other part of the body.

During a mastectomy, a surgeon often removes skin as well as breast tissue, leaving the remaining chest tissues flat and tight. To create a breast-shaped space for a reconstructive implant, a tissue expander is sometimes used as described above.

In any event, it should be appreciated that locating the fill valve on a prosthesis such as a tissue expander or adjustable implant requires considerable practitioner skill. Attempts to make products which facilitate this include the development of various products having structure, for example, embedded magnets or a raised ring, for assisting physicians in locating the valve.

It has also proven difficult to develop a flexible protective material that is effective as a puncture resistant material while also being safe for implantation in the body. A puncture resistant material used as a component of a breast implant or tissue expander would ideally be sufficiently flexible such that the implant could still be folded or rolled and inserted through a small incision while also providing resistance to needle punctures aimed at inflating the implant/expander to its final size.

Bark et al., U.S. Pat. No. 5,066,303, discloses a self-sealing tissue expander with a shell having a flowable sealing material. According to Bark et al., fluid infusion into the shell can be done directly through the shell, without the need for a fluid entry port.

Schuessler, U.S. patent application Ser. No. 12/543,795, filed on Aug. 19, 2009, the entire disclosure of which is incorporated herein by this specific reference, discloses a fluid filled implant including a self-sealing shell.

It has also proven difficult to develop a flexible protective material that is effective to protect the tubing leading from an implantable access port. Such an access port may be used as part of an implantable gastric banding system, for example, a system using the LAP-BAND® (Allergan, Inc., Irvine, Calif.) gastric band or the LAP-BAND AP® (Allergan, Inc., Irvine, Calif.) gastric band.

There is a need for improved temporary tissue expanders, more permanent adjustable implants, and other inflatable prostheses. In addition, there is a need for a flexible protective material that is effective to protect the tubing leading from an implantable access port. The present invention addresses these needs.

### SUMMARY

The invention relates, in part, to expandable prostheses, for example, implants and tissue expanders, and in particular to implantable temporary tissue expanders as well as more permanent mammary prostheses. The invention additionally relates to protective materials that may be used to protect access port tubing from puncture.

Accordingly, the present invention provides, in part, implants, for example, but not limited to tissue expanders and more permanent prostheses, for example, those implantable in a breast, and methods of making the same. The present invention provides inflatable prosthetic implants, components thereof and methods of making the same. In one embodiment of the invention, inflatable prosthetic implants are provided which include, as a component of such implants, flexible, puncture resistant materials.

In another embodiment of the invention, inflatable implants or prostheses, for example, tissue expanders and adjustable implants are provided which generally comprise a puncturable, self-sealing anterior portion, or shell, a puncture resistant posterior portion substantially opposing the anterior portion, and a fillable cavity defined between the anterior portion and the posterior portion.

It is to be appreciated that the terms “implant” “prosthesis” and “tissue expander” as used herein are intended to encompass permanent implants, including adjustable implants, as well as relatively temporary tissue expanders, and components, for example, shells, of such implantable devices.

In one embodiment of the invention, a method of making an inflatable device or prosthesis, suitable for implantation in a mammal, is provided wherein the method generally comprises the steps of providing a plurality of mesh segments, positioning the plurality of segments on a curved molding surface, applying a fluid elastomeric material to the molding surface with the segments positioned thereon, and allowing the elastomeric material to set to form a flexible shell having an open end, the shell including the fabric segments embedded within the set elastomer, and the shell being useful as a component of an inflatable prosthesis. The step of positioning may include substantially entirely covering the molding surface with the mesh segments, for example, in a manner such that the mesh segments overlap one another. The method further comprises the step of sealing the open end of the elastomeric shell, for example, by providing a puncture resistant member and sealing the puncture resistant member to the open end of the elastomeric shell.

In one embodiment, the mesh segments comprise a non-stretchable mesh fabric, for example, a substantially non-expanding polyester fabric mesh. In another embodiment, the mesh segments comprise a stretchable mesh fabric.

The method may further comprise the step of applying a tacky material to the curved molding surface prior to the step of positioning the mesh. The tacky material may be a fluid elastomeric material, for example, a silicone dispersion.

In another embodiment, the method comprises pre-shaping, for example, thermoforming, a mesh element, from a two-dimensional sheet into a three dimensional “sock” having the general shape of the molding surface. The method includes positioning the pre-shaped mesh element onto the molding surface, applying a fluid elastomeric material to the molding surface with the pre-formed mesh positioned thereon, and allowing the elastomeric material to set to form a flexible shell having an open end, the shell including the preformed mesh embedded within the set elastomer, and the shell being useful as a component of an inflatable prosthesis.

In another embodiment of the invention, an inflatable prosthesis made by the methods described herein is provided.

Further, in another embodiment, an inflatable prosthesis generally comprises an interior shell defining an inflatable chamber, an exterior shell comprising a silicone-based elastomer material having a mesh embedded therein, a gel separating the interior shell and the exterior shell, and a puncture resistant member forming a base of the prosthesis.

In yet another embodiment of the invention, a method of making a needle guard for an inflatable prosthesis suitable for implantation in a mammal is provided. The method generally comprises the steps of providing a first layer of puncture resistant members, for example, elongated slats, providing a second layer of puncture resistant members such that the second layer of members overlies and is offset from the first layer of members, molding or otherwise applying a flexible material to the first layer of members and the second layer of slats to form a device useful as a needle guard for an inflatable prosthesis. The step of applying or molding includes coupling the members to, for example, encasing the members within the flexible material.

In one embodiment, the members are elongated slats, and the slats of the first layer are substantially parallel to the slats of the second layer. The slats may be made of any suitable puncture resistant material, for example, a material selected from a group of materials comprising acetal, nylon, and polycarbonate. In some embodiments, the slats are made of a metal, for example, stainless steel, aluminum or titanium. The slats may be individual, separate elements that are cut from a sheet of material using any suitable means such as laser cutting. In other embodiments, at least one of the first layer of slats and the second layer of slats comprises a single, undivided sheet of material having grooves defining the adjacent slats.

In some embodiments, the step of applying a flexible material comprises applying an elastomeric sheet between the first layer of slats and the second layer of slats, for example, applying an uncured elastomeric sheet between the first layer of slats and the second layer of slats, and subsequently curing the sheets.

Alternative to the first and second layers of slats, a puncture-resistant fabric may be used, for example, in conjunction with an elastomeric layer, to form a suitable needle guard.

In one embodiment of the invention, a method for making an inflatable prosthesis suitable for implantation in a mammal is provided, wherein the method comprises providing a needle guard made by a method of the invention as described elsewhere herein and securing a flexible, inflatable shell to the needle guard.

In another embodiment of the invention, an inflatable prosthesis is provided generally comprising a flexible shell forming an anterior surface of the prosthesis, wherein the needle guard forms at least a portion of a posterior surface of the prosthesis, and comprises an elastomer portion and a first layer of puncture resistant slats embedded in the elastomer portion.

The needle guard may further comprise a second layer of puncture resistant slats. In some embodiments, the second layer of slats is offset from the first layer of slats.

In yet another embodiment of the invention, flexible, resilient puncture resistant assemblies are provided, the assemblies being, useful as components of surgical implants, for example, but not limited to, needle guards as components of inflatable implants that are accessed with a needle and a syringe. Such implants for which the present materials are useful include inflatable tissue expanders. Other implants that can benefit from the present invention include fluid access ports which include a fluid reservoir and a needle penetratable septum. In these and other implantable devices, puncture resistant or puncture proof assemblies of the invention can be highly beneficial, for example, as a means for preventing a needle tip from penetrating other areas of the device that are not intended to be punctured. For example, a needle guard assembly may serve to protect a tube leading from an implantable access port from being punctured by a syringe needle. Other beneficial uses for the present assemblies will become more apparent upon reading the present specification, and are considered to be included within the scope of the invention.

For example, puncture resistant assemblies are provided which are flexible and/or formable into desired configurations.

In some embodiments, puncture resistant assemblies are provided which are both flexible and resilient. Some of the present assemblies have the characteristic of shape memory, such that after being rolled or folded, they can resume an original shape or configuration. This embodiment of the invention is particularly, but certainly not exclusively, useful for application in a surgical environment, in which the assembly may be in the form of a puncture proof material is rolled or folded into a narrow configuration, thereby enabling insertion thereof through a relatively small incision. Advantageously, some of the assemblies of the invention are structured to be able to automatically resume an original, pre-deformed shape, for example, automatically, once the material is at the desired implantation site.

In one embodiment of the invention, a puncture resistant assembly is provided which generally comprises a first composite guard, a second composite guard, and an intermediate layer securing the first and second composite guards together and/or containing the first and second composite guards.

Each of the first and second composite guards generally comprises an arrangement of puncture resistant elements or members and a flexible substrate on which the members are secured and positioned, generally in a spaced-apart relationship.

The members may be in the form of domes or plates. The members have a hardness effective to resist penetration, puncture or breakage upon forceful contact with a sharp surface, for example, a tip of a needle, an edge of a cutting implement such as a scalpel or knife, or the like. The members may be made of any suitable material, such as a hard moldable substance, for example, a high durometer elastomer, polymer or rubber. Other suitable materials include metals, ceramics, and alloys thereof.

The flexible substrate on which the members are disposed may comprise a fabric, mesh, film, elastomer, or other material.

Notably, the first composite guard and the second composite guard are disposed with respect to one another such that the arrangement of members of the first composite guard is offset or misaligned with respect to the arrangement of members of the second composite guard. In some embodiments, a third composite guard is provided. The third composite guard may be positioned with respect to the first and second composite guards such that the members of the third composite guard are misaligned with the members of at least one of the first and second composite guards.

Advantageously, the misaligned or overlapping members of the adjacent composite guards provide a puncture resistant, or puncture proof, area while not significantly sacrificing flexibility of the assembly as a whole. That is, the composite guards may be arranged such that there are no significant gaps between individual puncture resistant members. It can be appreciated that depending upon the use of the final assembly, there may be some gaps between members so long as the gaps are sufficiently narrow to resist or prevent penetration by the type of instrument that the assembly is intended to be protected against puncture from.

In any event, in some embodiments of the invention, the puncture resistant members of the composite guards may provide an area of protection that substantially entirely covers a first side of the needle guard assembly.

The assembly may further comprise an intermediate layer, for example, an elastomer, securing together the first and second composite guards such that the members maintain their offset relationship. The intermediate layer may be located between adjacent composite guards and may be bonded thereto. In one embodiment, the intermediate layer seals the flexible composite members together and encapsulates the composite guards. For example, the intermediate layer may be a fluid tight barrier containing the two or more layered composite guards. In some embodiments, the intermediate layer exhibits a springiness and resiliency or provides a shape memory characteristic to the assembly.

In another aspect of the invention, a method of making a needle guard assembly is provided wherein the method generally comprises the steps of providing first and second composite guards where each composite guard includes a layer of puncture resistant members secured to a flexible substrate and bonding the first composite guard with the second composite guard in such that the members of the first composite guard are misaligned with the members of the second composite guard. In some embodiments, the method includes the step of bonding a third composite guard to the second composite guard such that the members of the third composite guard are misaligned with the members of at least one of the first composite guard and/or the second composite guard.

In some embodiments, the method may comprise the step of providing an intermediate layer between the composite guards. In some embodiments, the method may comprise the step of encasing or encapsulating the composite guards in a fluid tight seal.

In one embodiment, an inflatable prosthesis is provided which comprises an inflatable portion including an interior shell, an exterior shell comprising a silicone-based elastomer material having a mesh embedded therein and a gel separating the interior shell and the exterior shell. The prosthesis further comprises a needle guard assembly comprising a first composite guard and a second composite guard, each composite guard including an arrangement of puncture resistant members and a flexible substrate having a first side on which the puncture resistant members are disposed in a spaced apart fashion. The first composite guard and the second composite guard are positioned such that the arrangement of puncture resistant members of the second composite guard are misaligned with the arrangement of puncture resistant members of the first composite guard. The needle guard assembly further comprises an intermediate layer disposed between and connecting the first composite guard with the second composite guard.

In one embodiment, a needle guard assembly to protect a tube leading from an access port is provided. The needle guard assembly protects the tube from puncture by an incoming syringe needle. The needle guard assembly may comprise a first composite guard and a second composite guard, each composite guard including an arrangement of puncture resistant members and a flexible substrate having a first side on which the puncture resistant members are positioned. The first composite guard and the second composite guard are positioned such that the arrangement of puncture resistant members of the second composite guard is misaligned with the arrangement of puncture resistant members of the first composite guard. The needle guard assembly further comprises an intermediate layer positioned between and connecting the first composite guard with the second composite guard. The needle guard assembly may further comprise a top layer and a bottom layer forming outer surfaces of the needle guard assembly.

Each and every feature described herein, and each and every combination of two or more of such features, is included within the scope of the present invention provided that the features included in such a combination are not mutually inconsistent.

### BRIEF DESCRIPTION OF THE DRAWINGS

The present invention may be more clearly understood and certain aspects and advantages thereof better appreciated with reference to the following Detailed Description when considered with the accompanying Drawings of which:

FIG. 1 is cross-sectional view of a tissue expander in accordance with an embodiment of the invention, the tissue expander shown as implanted in a breast of a human being;

FIG. 2 is magnified view of a portion of the expander shown in FIG. 1;

FIG. 3 is a cross-sectional view of another tissue expander in accordance with an embodiment of the invention;

FIG. 4 is a cross-sectional view taken along line 4-4 of FIG. 3;

FIGS. 4A and 4B are a simplified top view and cross sectional view, respectively, of a needle guard feature of the tissue expanders in accordance with an embodiment of the invention;

FIG. 5 is a cross-sectional view of another tissue expander in accordance with an embodiment of the invention;

FIG. 6 is a cross-sectional view of yet another tissue expander in accordance with an embodiment of the invention;

FIG. 7 is a cross-sectional view taken along line 7-7 of FIG. 6;

FIGS. 8-10 show steps useful in making some of the tissue expanders in accordance with an embodiment of the invention;

FIG. 11 is cross-sectional view of another inflatable prosthesis including a puncture resistant assembly in accordance with an embodiment of the invention;

FIG. 12 is an exploded view of the prosthesis shown in FIG. 11 in order to illustrate certain components of the puncture resistant assembly;

FIG. 13 is a top view of a composite guard which is a component of the puncture resistant assembly shown in FIG. 11;

FIG. 14 is a magnified view of a portion of the composite encompassed by line 14 of FIG. 13;

FIG. 15 is a cross-sectional view of the composite guard taken along line 15-15 of FIG. 14;

FIG. 16 is a cross-sectional view, similar to the view shown in FIG. 15, of an alternative composite guard in accordance with an embodiment of the invention;

FIG. 16a is a cross-sectional view, similar to the view shown in FIG. 15, of yet another composite guard in accordance with an embodiment of the invention;

FIGS. 17-19 illustrate steps useful in making some of the puncture resistant assemblies in accordance with an embodiment of the invention;

FIG. 20 illustrates a perspective view of a gastric banding system including a needle guard assembly, in accordance with an embodiment of the invention;

FIG. 21 illustrates a perspective view of an access port, tube, and needle guard assembly in accordance with an embodiment of the invention;

FIG. 22 illustrates a cross-sectional view of the needle guard assembly and tube shown in FIG. 21, taken along line 22-22 of FIG. 21;

FIG. 23 illustrates a perspective view of an access port, tube, and needle guard assembly in accordance with an embodiment of the invention;

FIG. 24 illustrates a cross-sectional view of the needle guard assembly and tube shown in FIG. 23, taken along line 24-24 of FIG. 23;

FIG. 25 illustrates a magnified view of a portion of the needle guard assembly encompassed by line 25 of FIG. 24;

FIG. 26 illustrates a perspective view of a clip engaged with an access port in accordance with an embodiment of the invention;

FIG. 27 illustrates a perspective view of the clip shown in FIG. 26, in accordance with an embodiment of the invention;

FIG. 28 illustrates a bottom view of the clip shown in FIG. 26, and a bottom view of the access port shown in FIG. 26, in accordance with an embodiment of the invention;

FIG. 29 illustrates a perspective view of a needle guard assembly having a flanged portion, in accordance with an embodiment of the invention;

FIG. 30 illustrates a perspective view of the needle guard assembly shown in FIG. 29, separated from an access port, in accordance with an embodiment of the invention;

FIGS. 31 and 32 illustrate sheets of material for use as composite guards in accordance with an embodiment of the invention;

FIG. 33 illustrates a top view of a composite guard in accordance with an embodiment of the invention;

FIG. 34 illustrates a perspective view of three composite guards and a frame in accordance with an embodiment of the invention;

FIG. 35 illustrates a top perspective view of three composite guards with a frame passing therethrough in accordance with an embodiment of the invention;

FIG. 36 illustrates a bottom perspective view of the three composite guards with a frame passing therethrough, as shown in FIG. 35;

FIG. 37 illustrates a perspective view of a needle guard assembly in accordance with an embodiment of the invention; and

FIG. 38 illustrates a magnified view of a portion of the needle guard assembly shown in FIG. 37.

### DETAILED DESCRIPTION

The present invention generally pertains to implantable inflatable devices and methods for making same, for example, devices such as soft fluid-filled implants, for example, but not limited to, permanent or temporary implants useful in breast reconstruction or breast augmentation procedures.

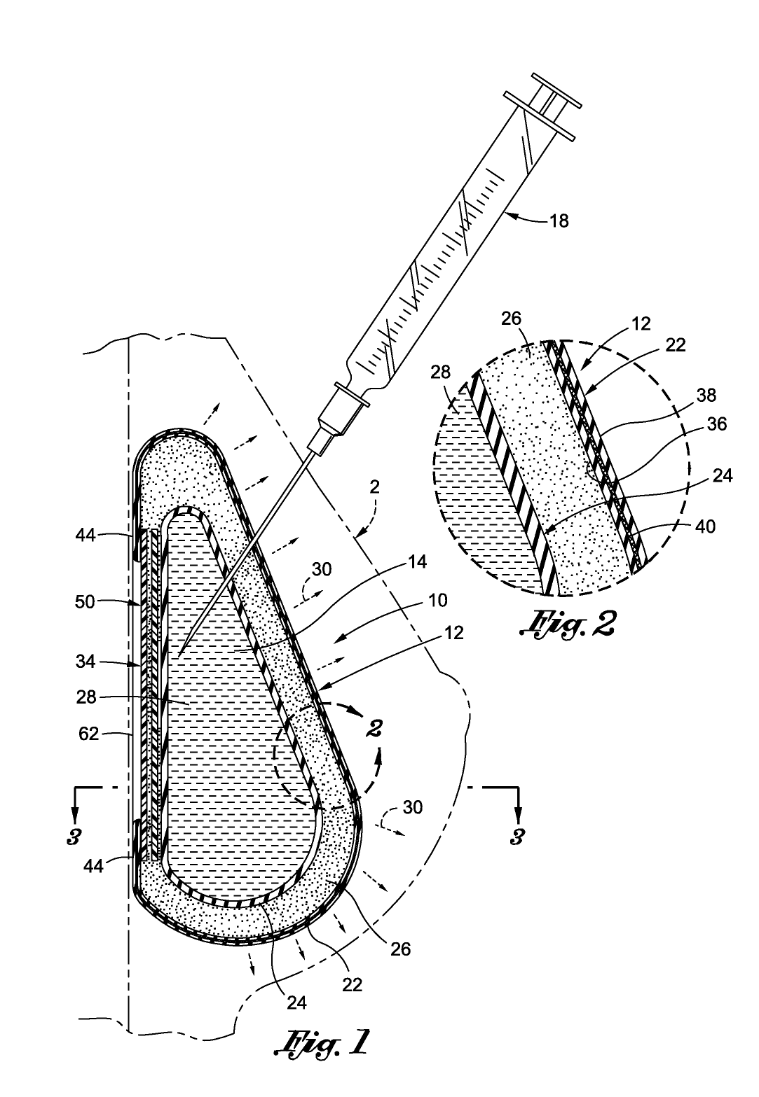

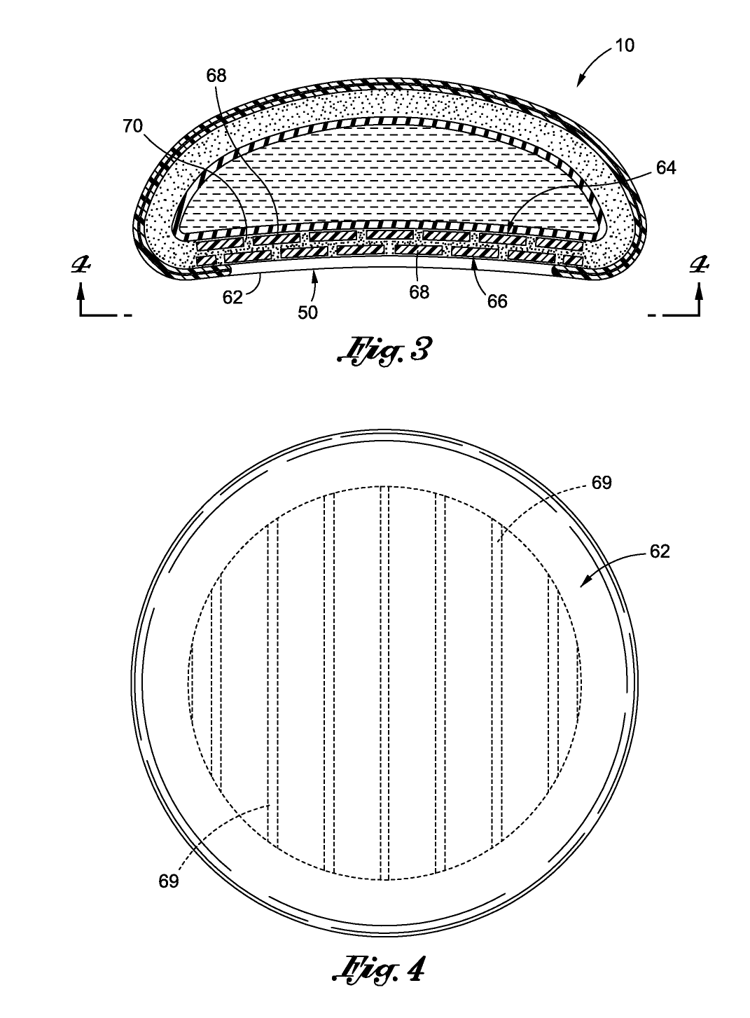

Turning now to FIG. 1, an inflatable device, in accordance with an embodiment of the invention, is shown generally at 10, as implanted in a human breast 2. The device 10 is being inflated with a suitable fluid, such as a saline solution 14, by means of a typical syringe 18.

The device 10 generally comprises an inflatable portion 12 comprising an outer shell 22, an inner shell 24 and an intermediate layer 26 therebetween. The inner shell 24 defines an inflatable cavity 28 (shown here as being filled with saline solution 14).

Inflation of the cavity 28 causes expansion of the device as shown by arrows 30. The device 10 further includes a posterior portion 34 that is generally resistant to expansion upon inflation of the cavity 28. The total volume of the device 10 is adjustable by introduction and removal of fluid into and from the fillable cavity 28.

The outer shell 22 of the device 10 may comprise at least one layer of elastomeric material, for example, a first layer 36 of elastomeric material and a second layer 38 of elastomeric material, and an additional layer of a different material, for example, a reinforcement layer 40 located between the first and second layers 36, 38 of the elastomeric material.

The elastomeric material may be a silicone elastomer such as a dimethyl silicone elastomer, for example, a substantially homogeneous dimethyl-diphenyl silicone elastomer. One composition useful in the present invention is described in Schuessler, et al., U.S. application Ser. No. 12/179,340, filed on Jul. 24, 2008, the disclosure of which is incorporated herein in its entirety by this specific reference. The elastomeric material may comprise a room temperature vulcanizing (RTV) or a high temperature vulcanizing (HTV) silicone from about 0.1-95 wt %, for example, about 1-40 wt %, for example, about 30 wt %. In an exemplary embodiment, the silicone-based fluid material is a high temperature vulcanizing (HTV) platinum-cured silicone dispersion in xylene.

The reinforcement layer 40 may comprise a mesh or fabric, for example, a synthetic polymer mesh or fabric, for example, a mesh or fabric made from poly (ethylene terephthalate) (PET), polypropylene (PP), polyurethane (PU), polyamide (Nylon), polyethylene (PE), any other suitable material, or combinations thereof.

In an exemplary embodiment, the outer shell 22 is made by dipping two or more layers of silicone-based elastomer over a conventional breast implant mandrel, followed by placement of a pre-fabricated 2 or 4-way stretchable “sock” of the reinforcing material layer 40, followed by two or more dips of the silicone-based elastomer. The reinforcing “sock” is able to take the shape of the mandrel and the fabric is trapped on both sides between the elastomer layers 36, 38. In this embodiment, the stretchable pre-shaped “sock” (which may form the reinforcing layer 40 of the outer shell 22) can be relatively easily mounted on the mandrel because of its flexibility and elasticity, making it easier to manufacture a reinforced shell with the intended shape and dimensions of the mandrel. The entire assembly forming the outer shell 22 is heated in an oven at a temperature and time suitable to cure the silicone.

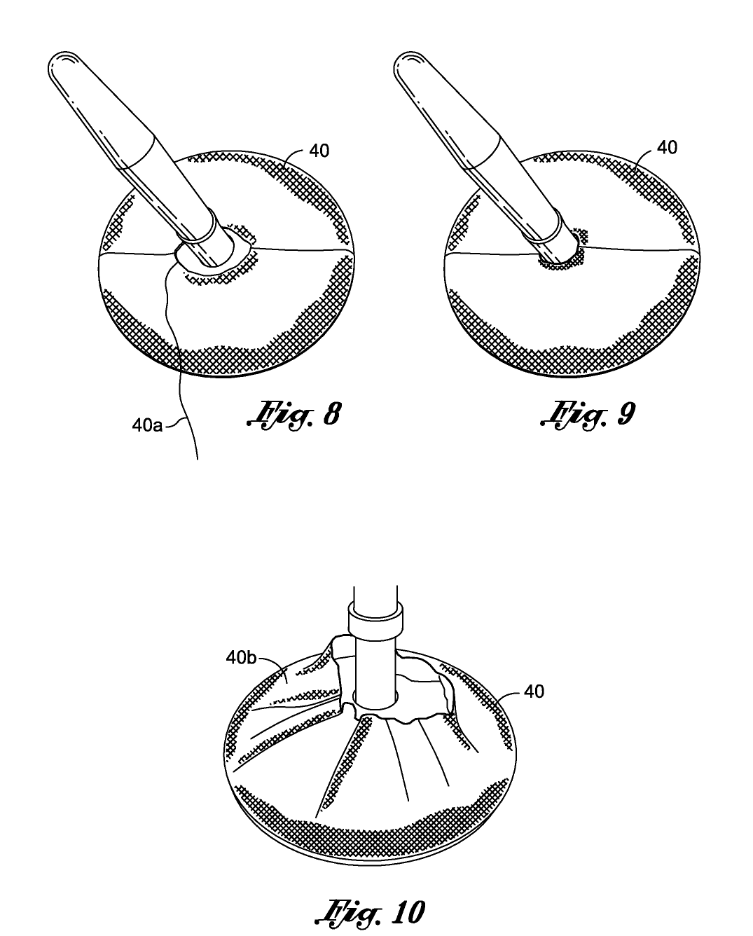

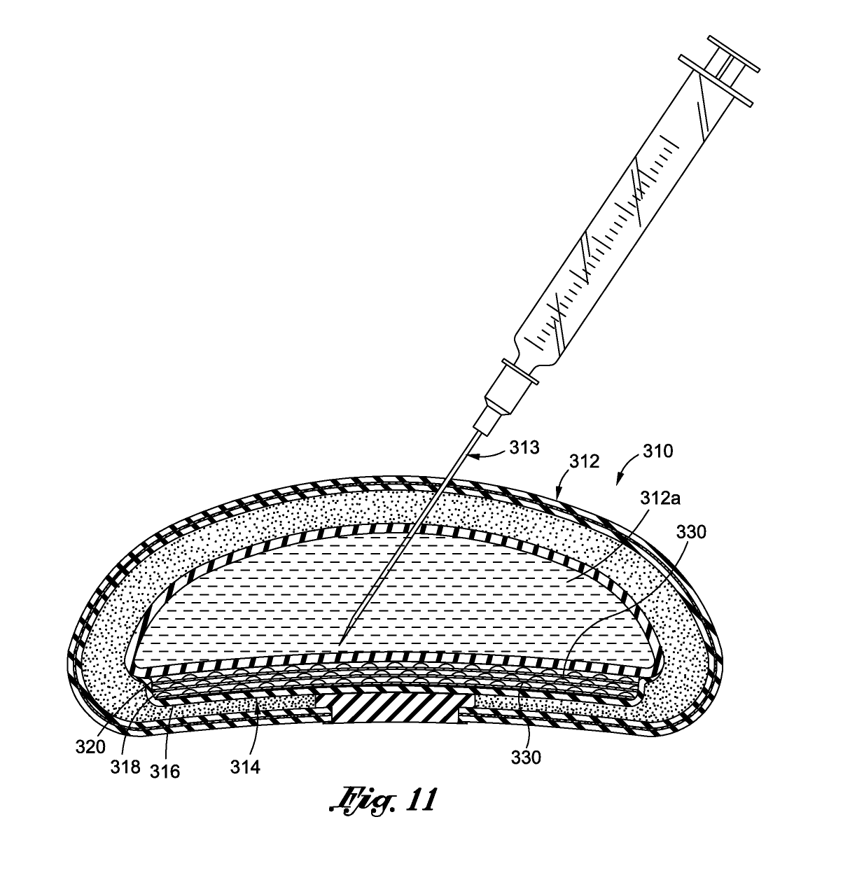

In one embodiment of the invention, the reinforcement layer 40 is provided by forming a “sock” by using a cinch 40a as illustrated in FIGS. 8 and 9. Alternatively, the reinforcement layer 40 is thermoformed into “sock” by placing a single sheet of suitable material, for example, a non-stretchable mesh, over a curved molding surface, for example, a mandrel, and gathering the mesh material at 40b, as shown in FIG. 10. The gathered mesh material is shaped, for example, thermoformed, to take on the 3-D shape of the mandrel.

Alternatively, rather than mesh sock, the reinforcement layer may comprise a plurality of fabric or mesh segments which are positioned on a mandrel or other curved molding surface. The segments may substantially entirely cover the molding surface. The segments may be positioned such that they overlap one another. The molding surface may first be contacted with a tacky material, for example, contacted with or coated with a silicone elastomer dispersion, to facilitate adherence of the segments thereto. An elastomeric material, such as an uncured silicone sheet or a silicone dispersion, is applied to the molding surface with the segments positioned thereon. The elastomeric material is allowed to set to form a flexible shell having an open end, the shell including the fabric or mesh segments embedded within the set elastomer, and the shell being useful as a component of an inflatable prosthesis.

Post-curing, the reinforced shell is removed from the mandrel, and another elastomeric shell (which forms the inner shell 24) is placed inside the first shell (which forms the outer shell 22). The inner shell 24 may be a typical unreinforced elastomeric shell, or alternatively may be made similarly to that described above with respect to the outer shell 22. The inner shell 24 may have the same or smaller size relative to the outer shell 22. The two shells 22, 24 are vulcanized close to their open base using, for example, a ring-shaped patch 44, thus forming an inter-shell compartment. The dual-shell assembly is mounted back on a mandrel. The size of the mandrel can be the same as the one used for the inner shell fabrication or slightly larger. The latter would result in a laterally stressed inner shell with potentially enhanced sealing properties.

In some embodiments of the invention, at least one of the inner shell 24 and the outer shell 22 comprises an elastomeric material comprising a substantially homogenous layer of a silicone elastomer comprising a polysiloxane backbone and having a minimum mole percent of at least 10% of a substituted or pendant chemical group that sterically retards permeation of said silicone gel through the layer. More specifically, in this embodiment, the silicone elastomer is a polydimethyl siloxane and the pendant chemical group is one of a phenyl group, for example, a diphenyl group or a methyl-phenyl group, a trifluoropropyl group, and mixtures thereof. Such materials are described in detail in Schuessler, et al., U.S. patent application Ser. No. 12/179,340, filed on Jul. 24, 2008, the entire disclosure of which is incorporated herein by this specific reference. This material may make up one or more layers of the shell(s) 22, 24.

After the inner shell 24 and the outer shell 22 are bonded together, a cavity formed therebetween is then filled with a material, for example, a flowable material, for example, a silicone gel. This may be accomplished using any suitable means known to those of skill in the art. In one embodiment, the gel is introduced through a reinforced silicone plug on the outer shell 22. The silicone gel between the outer and inner shells 22, 24, forms the intermediate layer 26. After filling, the assembly made up of the inner shell 24, the outer shell 22 and the intermediate layer 26, is cured, for example, by exposing the assembly to heat in an oven for a suitable length of time. The mandrel that defines the desired shape of the implant can be round or oval, with a lower or upper pole for optimal projection. Before sealing the implant with a patch, a needle guard element, such as that described and shown elsewhere herein, may be inserted and bonded to the inner shell 24 and/or the outer shell 22, to form the posterior portion 34 of the device.

It can be appreciated that the device 10, in the form of a tissue expander, once implanted in a patient, should be repeatedly accessed during the expansion process with percutaneous needle punctures, such as shown in FIG. 1. In some embodiments, the tissue expander devices are able to survive repeated puncturing and over-expansion to 200% by saline without leakage.

The device 10 can also be in the form of a more permanent mammary prosthesis, for example, an adjustable breast implant. The volume of the implant can be adjusted in situ by accessing the cavity 28 with a needle through the self-sealing anterior portion of the device 10. In some embodiments, the cavity 28 has a small volume relative to the gel portion 26, to provide a comfortable implant having the desirable qualities of a gel-filled implant with the advantages of being size-adjustable with saline.

The anterior surface of the device 10 is self-sealing and can be accessed for fluid communication. The mechanism of self-sealing is facilitated by a combination of the gel layer 26 and shell 22. After a void is created by a needle used to introduce filler (saline) into the implant 10, the gel layer 26 prevents the saline 14 from having a direct path to the exterior and the reinforcing mesh 40 enhances this property by physically constraining the gel from expansion under pressure exerted by the saline 14. The reinforcing materials 40 include, but are not limited to, meshes and fabrics made from PET, PP, PU, Nylon, etc. and combinations thereof. This invention features a novel manufacturing method for shaping the implant shell into 2-D and 3-D structures making it more convenient to manufacture and convert these reinforced structures into mammary prostheses.

In order to limit the depth of penetration of the needle, and also to give the medical professional feedback as to when the needle has reached the correct location for filling, conventional tissue expander devices sometimes include a rigid backing or needle stop behind the filling port in the posterior side of the device. Typically these needle stops are made of metals or very hard or thick plastics to prevent needle penetration through the injection site. By nature then, these needle stops are quite rigid and inflexible, can be uncomfortable, and can limit the collapsibility of the device which affects ease of insertion of the expander through the initial incision.

The posterior portion 34 of device 10 may comprise an improved needle guard 50. The needle guard 50 may comprise any suitable biocompatible polymer (e.g. PE, PP, PU, PET, PI, TPU, high durometer silicones, ABS etc.) that is strong enough to resist needle puncture. The needle guard 50 may comprise one or more layers 56 of puncture resistant material with or without an intermediate layer 58. In some embodiments, the needle guard 50 is structured so as to prevent, or substantially prevent, the device 10 from expanding toward the chest wall during inflation of the cavity 28.

For filling an implant of the present invention, a syringe coupled to a 21 g or smaller needle may be used. The needle may be introduced anywhere in the anterior portion of the implant, such that it reaches the needle guard 50, where it is prevented from penetrating further. The implant is then filled with saline or other liquids for tissue expansion. After removal of the needle, the assembly (e.g., the outer shell 22, the inner shell 24 and the intermediate layer 26) self-seals and prevents the implant from leaking.

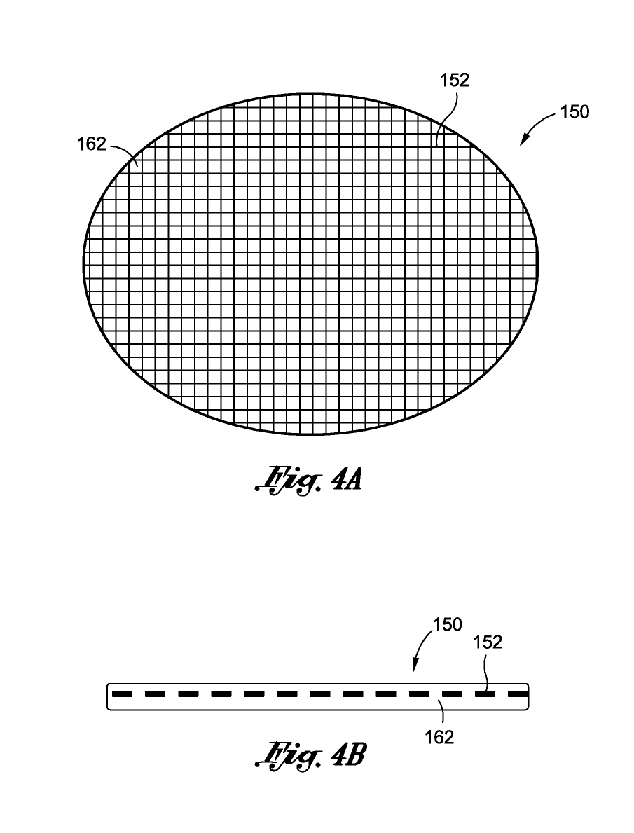

In FIGS. 3 and 4, the needle guard 50 may comprise an elastomer portion 62, and one or more layers of puncture resistant members coupled thereto. In the shown embodiment, members comprise elongated members, for example, slats 68 coupled to the elastomer portion 62.

In this case, the needle guard 50 comprises one or more layers of slats 68, for example, a first layer 64 of slats 68 and a second layer 66 of slats 68 coupled to the elastomer portion 62. As shown, the slats 68 of the first layer 64 overlap, or are offset from, the slats 68 of the second layer 66. For example, spacing between the slats 68 of the first layer 64 are aligned with slats of the second layer 66 and vice versa. The elastomer portion 62 may include grooves 69 or slots. The grooves may be aligned with the slats 68 to facilitate rolling or folding of the device 10.

The slats 68 extend across substantially the entire posterior portion 34 and are aligned substantially parallel to one another. This arrangement allows the device 10 to be rolled or folded in alignment with the slats 68 while the offset or overlapping positioning of the first and second layers 64, 66 provides protection in the event a needle enters the spacing 70 between adjacent slats 68.

Alternative to this arrangement, adjacent slats in each layer may overlap one another (not shown). The needle guard comprises overlapping but independent small pieces of rigid puncture-resistant material, and like the offset layers of slats 68 described and shown elsewhere herein, the overlapping configuration provide that there are no “line-of-sight” openings through which a needle can pass.

The slats 68 may be a polymer material. The slats 68 may be, for example, nylon, acetal, polycarbonate, or other suitable, biocompatible, puncture resistant or puncture-proof polymeric material. The slats 68 may be metal, for example, stainless steel, aluminum or titanium.

In various exemplary embodiments, the slats 68 may be between about 10 mm to about 100 mm or more in length, about 2 mm to about 30 mm in width, and about 0.2 mm to about 4 mm in thickness. The slats of other configurations and dimensions suitable for achieving the desired flexibility of the needle guard 50 may also be used. Such variations of materials and dimensions are considered to fall within the scope of the present invention. In one embodiment, the slats 68 have a thickness of about 2 mm and the needle guard 50, including first and second layers 64, 66 of the slats 68 and elastomer material therebetween, has a total thickness of about 5.0 mm or less.

The slats 68 may be formed by laser cutting same from a sheet of material. Alternatively, the slats 68 may be defined by grooves in a single sheet of material. In this specific example, the 2 layers of parallel slats of puncture-resistant plastic about 0.25″ wide and with about 0.05″ open space between each slat. The layers are offset from each other so that the open space of one slat layer is centered on the middle of a slat in the layer below. All the slats are encapsulated in a soft flexible material like silicone. The open space between the slats gives the whole assembly flexibility to be readily folded or rolled up even though the plastic itself is rigid and resistant to extensive bending. Other shapes and layering designs of independent pieces of puncture resistant materials would provide the needle stop with more and different degrees of bending and folding capability.

The rigid or semi-rigid material forming the slats can be thermoplastics such as acetal, nylon, polycarbonate, and others; or thin metals such as stainless steels, aluminum, or titanium. The use of plastics can be advantageous in that the entire device 10 can be made to be MRI compatible.

In a similar embodiment of the invention, thin elastomeric films (0.25-1 mm) made of materials resistant to needle puncture may be used as a component of the needle guard portion of the implant. In some embodiments, such films can be provided with grooves in their design to allow folding/unfolding during insertion. The films may be attached to the shell using adhesives or alternatively may be are encapsulated in silicone.

In another embodiment, rather than independent slats 68, one or more layers of flexible “slat sheets” are provided. In this embodiment, adjoining slats can be made by starting with readily available sheets of the desired plastic of the appropriate thickness. Parallel, adjacent slats are created by laser cutting through the plastic to create the desired spacing between the slats but not all the way to the edges of the plastic sheet, thereby leaving a material, for example, a border that holds all the slats together. In this way, the pre-cut slats can still be handled as one piece and therefore maintain the desired spacing and orientation. In one embodiment, two of these pre-cut plastic “slat sheets” are alternately layered between 3 sheets of silicone. After curing the silicone, a die cutter of the desired shape of the needle stop can cut within the borders of the pre-cut slats to stamp out the finished needle stop that now has many unconnected slats each independently encased in silicone.

Alternatively still, the pre-cut slat sheets can be held in the desired orientation in a mold and silicone can be injected and cured around them. Additional assembly steps can include creating a silicone border around the needle stop that would assemble to the expander envelope, texturing or adding features to the needle stop surface, or shaping the needle stop assembly so that it has a concave exterior to better fit the chest wall anatomy in the case of a breast tissue expander.

Turning to FIGS. 4A and 4B, yet another variation of a needle guard 150 is provided, similar to the needle guard 50, except that rather than the slats 68, one or more layers of a puncture resistant mesh 152 are provided. The needle guard 150 may be substantially identical to the needle guard 50 described above, with one or more differences being as follows.

In the shown exemplary embodiment, the needle guard 150 comprises one or more layers of mesh 152, for example, a single layer of mesh 152 coupled to, for example, embedded in, the elastomer portion 162. In other embodiments, not shown, two or more layers of mesh are provided, wherein fibers or cords making up the mesh, in adjacent layers of mesh, overlap one another. For example, interstices or a spacing between a mesh fiber of a first layer of mesh aligns with the mesh fiber of a second layer of mesh, and vice versa. Alternatively, a single layer of mesh is provided with interstices between fibers being sized to prevent needle penetration therethrough.

Flexibility of the mesh 152 and the elastomer portion 162 allow the entire implant device to be rolled or folded upon insertion into a breast cavity through a small incision.

The mesh 152 may be a polymer or a metallic material. The mesh may be, for example, a polymer such as nylon, acetal, polycarbonate, or other suitable, biocompatible, puncture resistant or puncture-proof material. The mesh 152 may be metal, for example, stainless steel, aluminum or titanium.

It should be appreciated that in many of the embodiments of the present invention, the needle guard making up the posterior portion of the implant comprises puncture resistant members arranged in an overlapping configuration to provide no “line-of-sight” openings through which a needle can pass. These puncture resistant members can be variously configured and arranged to achieve this goal.

In a preferred embodiment, it is desirable for the needle stop to be flexible for insertion yet rigid to resist needle puncture. To prevent movement of the needle guard inside the device, the needle stop material may be adhered, fused or vulcanized to the posterior of the implant or the patch. For this purpose, the needle guard may be dipped in silicone that is then heat cured, such that the needle guard is covered by a silicone sheath. This silicone sheath is vulcanized to the silicone patch or posterior of the implant, to prevent movement of the guard inside the implant.

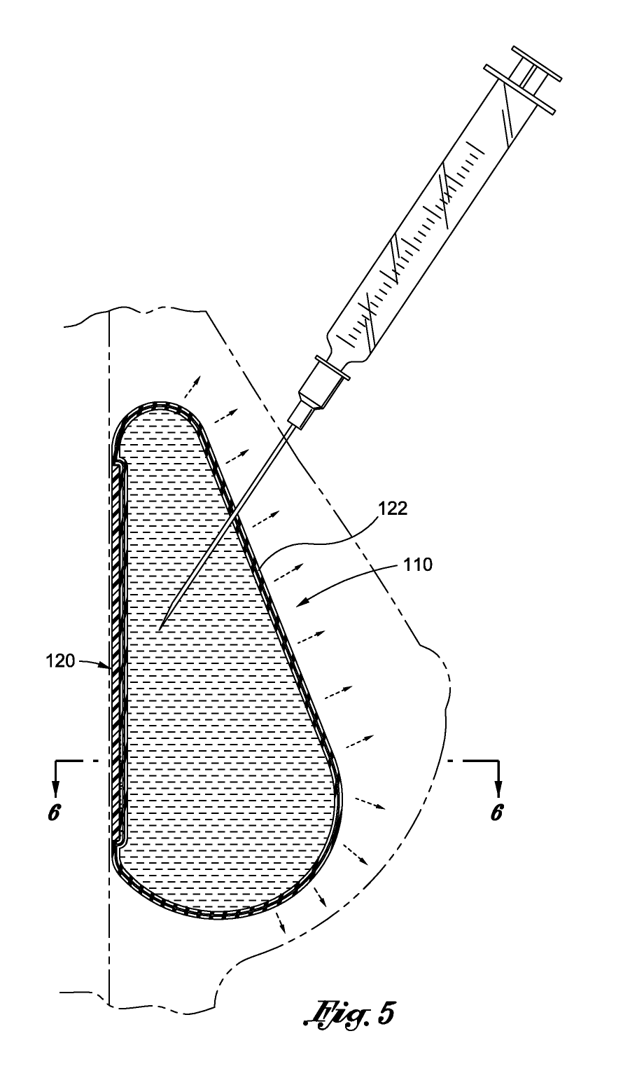

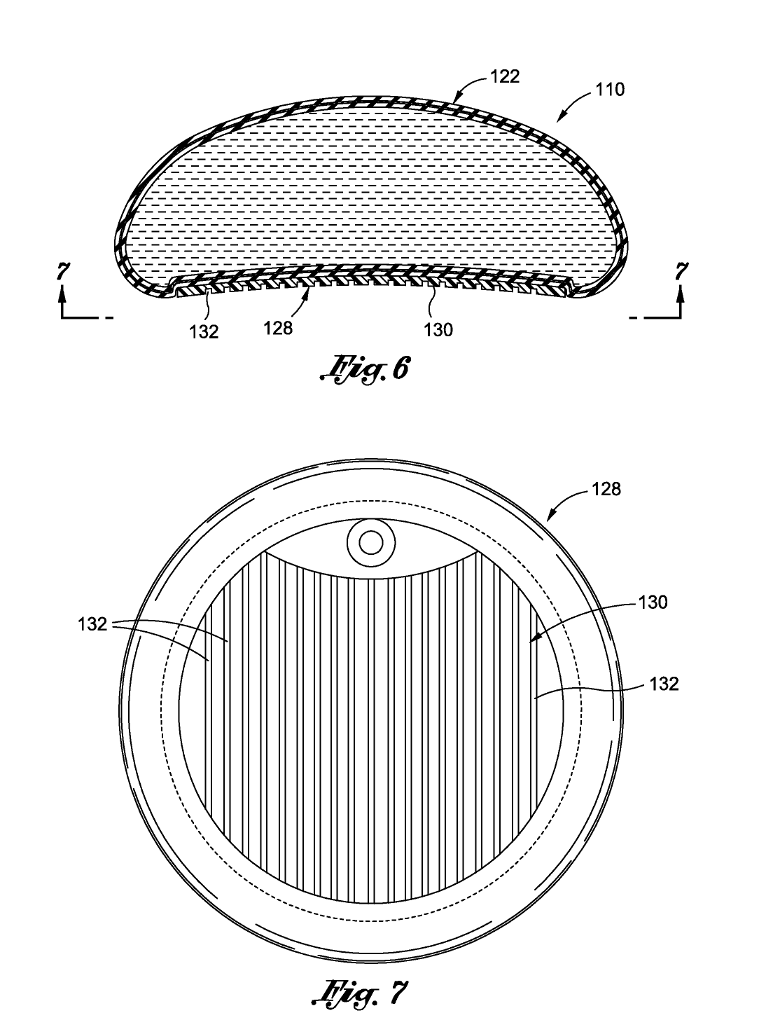

Another device 110 in accordance with the invention is shown in FIGS. 5-7. The device 110 may be substantially identical to the device 10 except that the device 110 does not include an inner shell 24 or an intermediate layer 26. The device 110 comprises a self-sealing outer layer 122. The self-sealing outer layer 122 may be identical to the layer 22 of the device 10. Further, rather than the needle guard 50, the device 110 comprises the needle guard 128 which comprises a puncture resistant elastomeric member 130 having grooves 132 for facilitating rolling or folding of the device 110 during insertion.

Turning now to FIGS. 11-16a, another device, for example, an inflatable implant, in accordance with the invention is shown generally at 310. The implant 310 may be identical to the implant 10 shown in FIG. 3, with the primary difference being that instead of the needle guard 50 made up of layers of slats as described elsewhere herein, the implant 310 includes a puncture resistant material 314 as shown and now described.

The device 310 includes an inflatable portion 312, and a puncture resistant assembly 314.

The device 310 is expanded or inflated (or deflated) by insertion of a needle 313 (FIG. 11) through the inflatable portion 312 (which may be identical to the inflatable portion 12 of the device 10) and introduction of fluid into a cavity 312a. Instead of the inflatable portion 12, it can be appreciated that the inflatable portion 312 can include any suitable structure, including an elastomeric bladder having an access port with a needle penetratable septum, or may be made partially or entirely of a puncturable, but self sealing material. Some suitable self sealing materials are described, for example, in U.S. patent application Ser. No. 12/543,795, filed on Aug. 19, 2009, the entire specifications of which are incorporated herein by this reference.

In order to prevent the needle 313 from undesirably penetrating through the device 310, the device is equipped with an assembly 314.

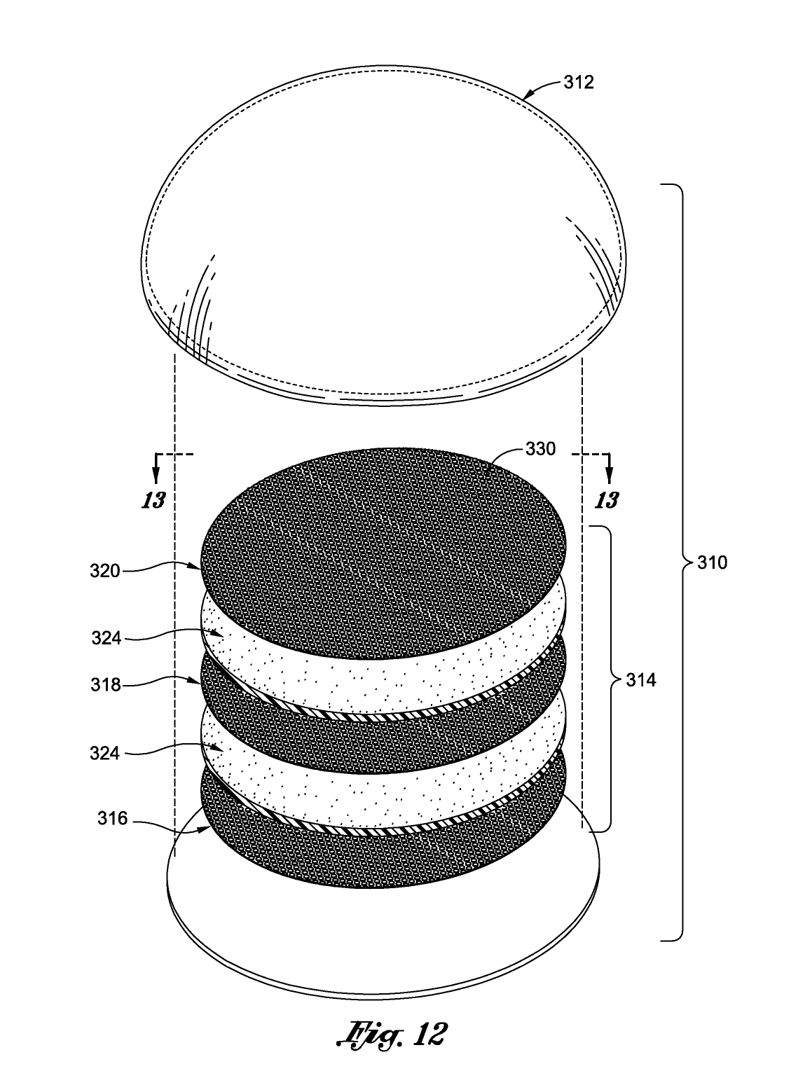

Referring now to FIG. 12, the assembly 314 generally comprises a first composite guard 316 and a second composite guard 318. In the shown embodiment, the assembly 314 further includes a third composite guard 320. In other embodiments, only two composite guards or more than three composite guards are provided. An intermediate layer 324 is provided between adjacent guards, for example, between the guard 316 and the guard 318, and likewise, between the guard 318 and the guard 320.

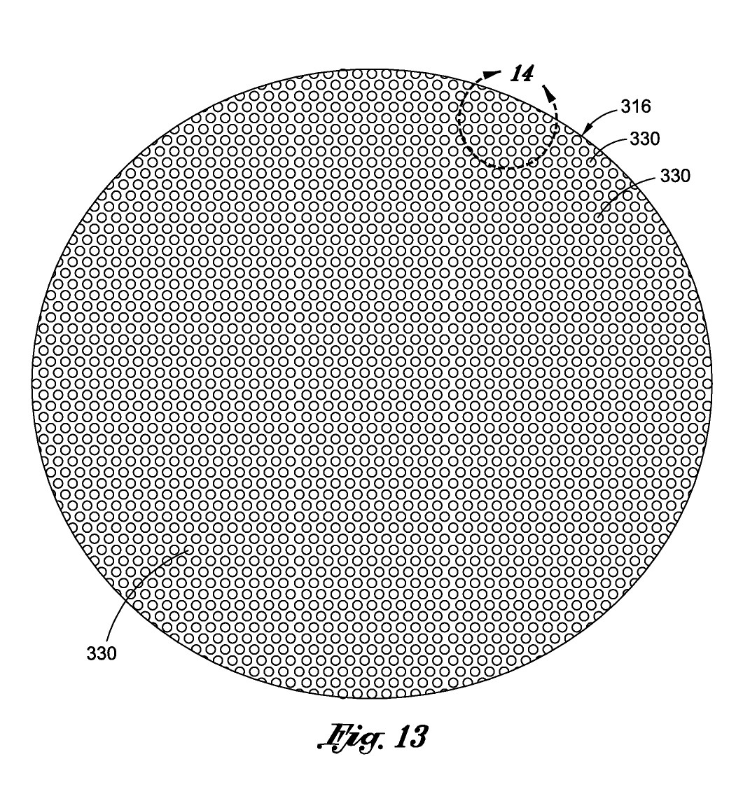

Turning now as well to FIGS. 13 and 14, each of the composite guards 316, 318, 320 includes a plurality of, for example, an arrangement, array, or pattern of, puncture resistant members 330, and a flexible substrate 332 having a first side on which the puncture resistant members 330 are disposed in a generally spaced apart fashion.

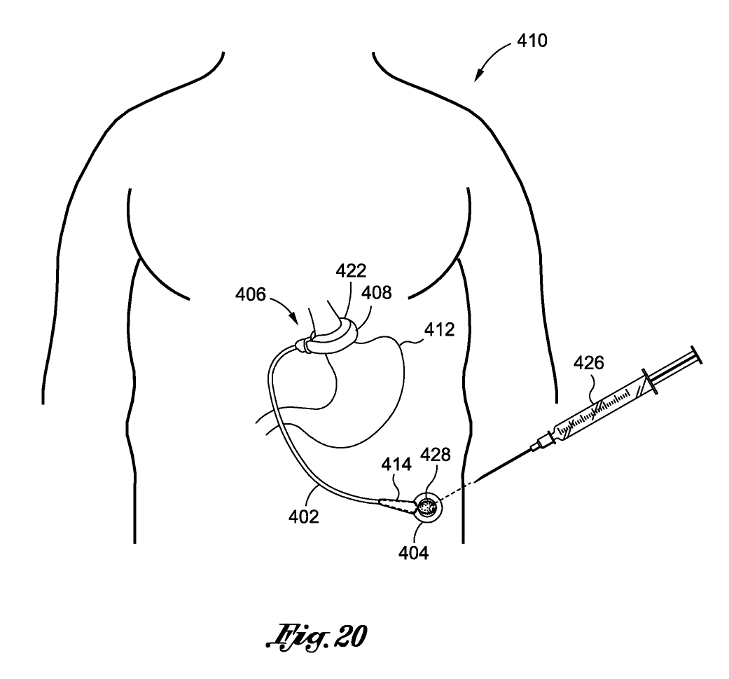

As can be perhaps best appreciated from FIG. 11 (and FIG. 19), the first composite guard 316 and the second composite guard 318 are positioned such that the arrangement of puncture resistant members 330 of the second composite guard 318 are misaligned with the arrangement of puncture resistant members 330 of the first composite guard 316. Similarly, the second composite guard 318 and the third composite guard 320 may be positioned such that the arrangement of the puncture resistant members of the third composite guard 320 are misaligned with the arrangement of puncture resistant members of at least one of the first composite guard 316 and the second composite guard 318. Thus, accordingly, the composite guards 316, 318, 320 are arranged relative to one another such that there are no straight line open spaces, or substantial gaps, between the members 330 to allow a needle or sharp implement to penetrate entirely through the assembly 314. Yet, advantageously, the assembly 314 as a whole may be quite flexible in that the substrate 332 on which the spaced apart 330 members are disposed is supple, flexible and/or bendable.

Turning specifically to FIG. 12, the intermediate layer 324 may comprise a flexible, connecting material which is effective to couple or bond the first composite guard 316 with the second composite guard 318, and the second composite guard 318 with the third composite guard 320. As shown in FIG. 12, the intermediate layer 324 is positioned between the arrangement of the puncture resistant members 330 of the first layer 316 and the flexible substrate 332 of the second layer 318, and another intermediate layer 324 is positioned between the arrangement of the puncture resistant members 330 of the second layer 318 and the flexible substrate 332 of the third layer 320.

The composite guards 316, 318, 320 may be identical to one another, and for the sake of simplicity, only the first composite guard 316 will now be described, with the understanding that, in the shown embodiment, what is described for the first composite guard 316 is also applicable to the second composite guard 318 and the third composite guard 320.

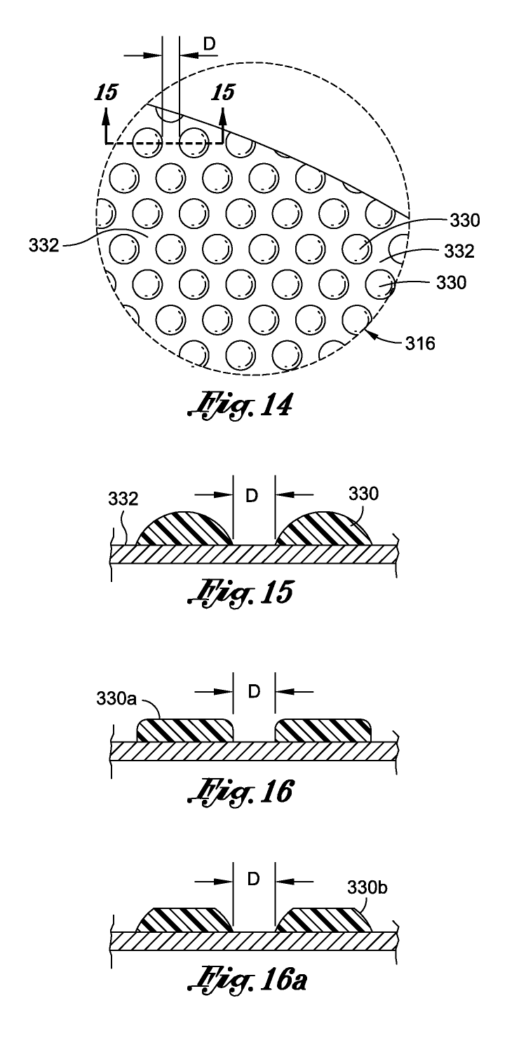

The members 330 may be any suitable shape. In FIG. 15, the members 330 are somewhat dome shaped with rounded surfaces. In other embodiments, the members 330a may be planar as illustrated in FIG. 16. Alternatively still, the members 330b may include both rounded surfaces and planar or flat surfaces, such as the members 330b which are dome shaped with a flat upper surface, as illustrated in FIG. 16a.

The members 330 have a thickness of between about 0.1 mm and about 1.0 mm, for example, a thickness of between about 0.2 mm and about 0.5 mm. The members 330 have a spacing D of between about 0.2 mm and about 0.5 mm. The members 330 have a diameter of between about 0.5 mm and about 2.0 mm, for example, a diameter of about 1.5 mm.

In some embodiments, the guard 316 includes between about 50 and about 1000 members per square inch (psi), for example, about 400 psi.

In a specific embodiment, the guard 316 include about 400 members psi, each having a diameter of about 1.5 mm and each being spaced apart about 0.2 mm.

The members 330 (and 330a and 330b) are made of a suitable puncture resistant material, such as an epoxy, polymer, rubber, ceramic or metal, or suitable combination or alloy thereof. For some applications, suitable materials include polyethylene (PE), polypropylene (PP), polyurethane (PU), polyethylene terephthalate (PET), polycarbonate (PC), polyisoprene (PI), thermoplastic urethanes and thermoplastic polyurethanes (TPU), high durometer silicones, acrylonitrile butadiene styrene (ABS), etc. In some embodiments, the members 330 are made of material such as acetal, nylon, and polycarbonate. In some embodiments, the members 330 are made of a metal, for example, stainless steel, aluminum, titanium, or other metal. The members 330 may be made of any other material specifically indicated to comprise the puncture resistant members 330 in this application, including any materials discussed in relation to the members 430, 436, 530, 536, 550 shown in FIGS. 22 and 24-25, and the members 1030, 1036, 1050 shown in FIG. 38.

The flexible substrate 332 may comprise a mesh, film, fabric, elastomer, or other suitable material. The flexible substrate 332 may be made of any other material specifically indicated to comprise the flexible substrate 332 in this application, including any materials discussed in relation to the flexible substrates 432, 434, 532, 534, 552 shown in FIGS. 22 and 24-25, and the substrates 1032, 1034, 1052 shown in FIG. 38.

The intermediate layer 324 may be a polymer, for example, an elastomeric polymer, for example, a silicone elastomer, for example, a low durometer silicone rubber. The intermediate layer 324 may be made of any other material specifically indicated to comprise the intermediate layer 324 in this application, including any materials discussed in relation to the intermediate layers 424, 524, 525 shown in FIGS. 22 and 24-25, and the intermediate layers 1024, 1025 shown in FIG. 38.

In some embodiments, the assembly 314 has a resiliency or a shape memory such that it will restore from a folded or rolled configuration to an original, different configuration. The original configuration may be a generally flat or planar configuration. This may be provided by using a suitable intermediate layer material, such as a silicone elastomer, that has a shape memory characteristic.

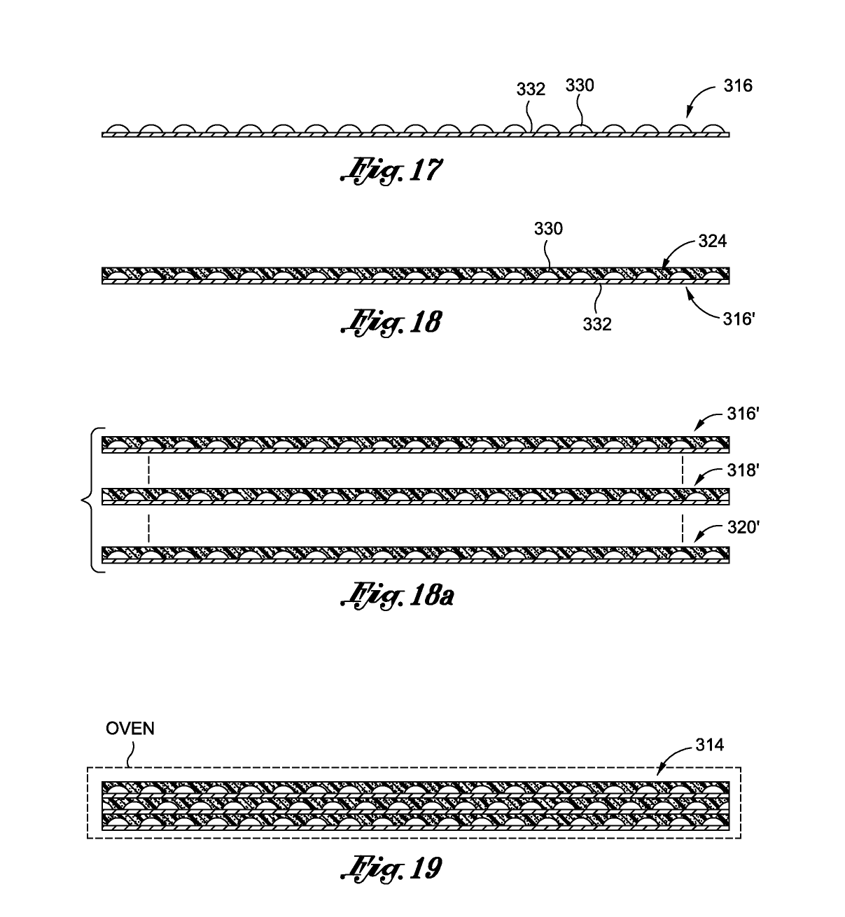

Assembly of the guard assembly 314 may be accomplished as follows and as shown in FIGS. 17-19.

Turning now to FIG. 17, the guard 316, generally comprising the members 330 and the substrate 332, is made by any suitable method, including stencil printing, for example, using equipment and processes used in surface mount technology/PCB fabrication. Other processes that can be used to make the guard 316 include micro-dot dispensing and printing, and laser etching. Other suitable methods will be known to those skilled in the art. Other suitable methods include those discussed in relation to the formation of the composite guards 416, 418, 516, 518, 520 shown in FIGS. 22 and 24-25.

Turning to FIG. 18, the intermediate layer 324 may be formed as follows. A suitable material, for example, a sheet of uncured silicone, is placed on one side of the guard 316, for example, on the side having the members 330 and the substrate 332. The sheet is then subjected to curing conditions to cause the sheet to adhere to the members 330, forming the intermediate layer 324 thereon. In the presently described example embodiment, this step is done three times, with three separate guards 316, 318, 320, to form the components 316′, 318′ and 320′ of assembly 314. (See FIG. 18a).

The assembly 314 is then placed in an oven or otherwise subjected to further curing conditions to seal the assembly 15 components together such as shown in FIG. 19.

FIG. 20 illustrates an embodiment of the present invention, in which a needle guard assembly 414, having a similar construction as the needle guard assembly 314 discussed in relation to FIGS. 11-19, is utilized to protect a tube 402 used in conjunction with an implantable access port 404. In the embodiment shown in FIG. 20, the tube 402, the access port 404, and the needle guard assembly 414, are used in an implantable gastric banding system 406, including a gastric band 408 configured to form a loop around a portion of a patient's 410 stomach 412 to form a stoma. The gastric band 408 may have a composition as described in Birk, U.S. Pat. No. 7,811,298, the entire disclosure of which is incorporated herein by this specific reference.

The gastric banding system 406 is used for the treatment of obesity. The gastric band 408 is preferably wrapped around the cardia, or esophageal junction of the stomach 412, to restrict the flow of food passing from the upper portions of the patient's 410 stomach 412 to the lower portions of the patient's 410 stomach 412. The restricted flow of food enhances the satiety signals sensed by the patient 410, which desirably reduces food consumption of the patient 410, which hopefully causes the patient 410 to lose weight.

Over time, a physician may need to adjust the degree to which the gastric band 408 constricts the patient's 410 stomach 412. As such, the gastric band 408 may include an inflatable portion 422, which comprises an inflatable cuff that wraps around the patient's 410 stomach 412. The inflatable portion 422 may be filled with fluid. The amount of fluid in the inflatable portion 422 defines the degree to which the gastric band 408 constricts the patient's 410 stomach 412 (e.g., a greater amount of fluid in the inflatable portion 422 will increase the constriction of the patient's stomach). A physician may adjust the amount of fluid in the inflatable portion 422 via the access port 404.

The access port 404 is preferably fixed subcutaneously within the patient's body, and is preferably fixed to body tissue including the patient's 410 interior muscle wall. The tube 402 conveys fluid to and from the inflatable portion 422, from the access port 404. One end of the tube 402 couples to the access port 404, and the other end of the tube 402 couples to the inflatable portion 422 of the gastric band 408.

A physician inserts a syringe 426 needle into the patient's body to access the access port 404, and vary the amount of fluid in the inflatable portion 422 of the gastric band 408. Generally, the physician must attempt to locate a septum 428 of the access port 404 to pass the syringe 426 needle through the septum 428. The septum 428 must be penetrated by the syringe 426 needle to allow fluid to enter, or be removed from the access port 404. The physician will typically palpate the area around the access port 404 to locate the septum 428.

However, it may be difficult for the physician to properly locate the septum 428, because the access port 404 may be covered by many layers of the patient's 410 fat. Accordingly, it is possible the physician may not properly locate the septum 428, and may errantly insert the syringe 426 needle. The physician may contact a portion of the tube 402 leading from the access port 428 to the gastric band 408. The syringe 426 needle may puncture the tube 402, specifically the end of the tube 402 connected to the access port 404, and may cause fluid to leak from the gastric banding system 406. A surgical procedure may be necessary to repair the punctured tube 402, or replace the entire gastric banding system 406. The needle guard assembly 414 is intended to prevent this undesirable result, by shielding the end of the tube 402 connected to the access port 404, and protecting the tube 402 from puncture.

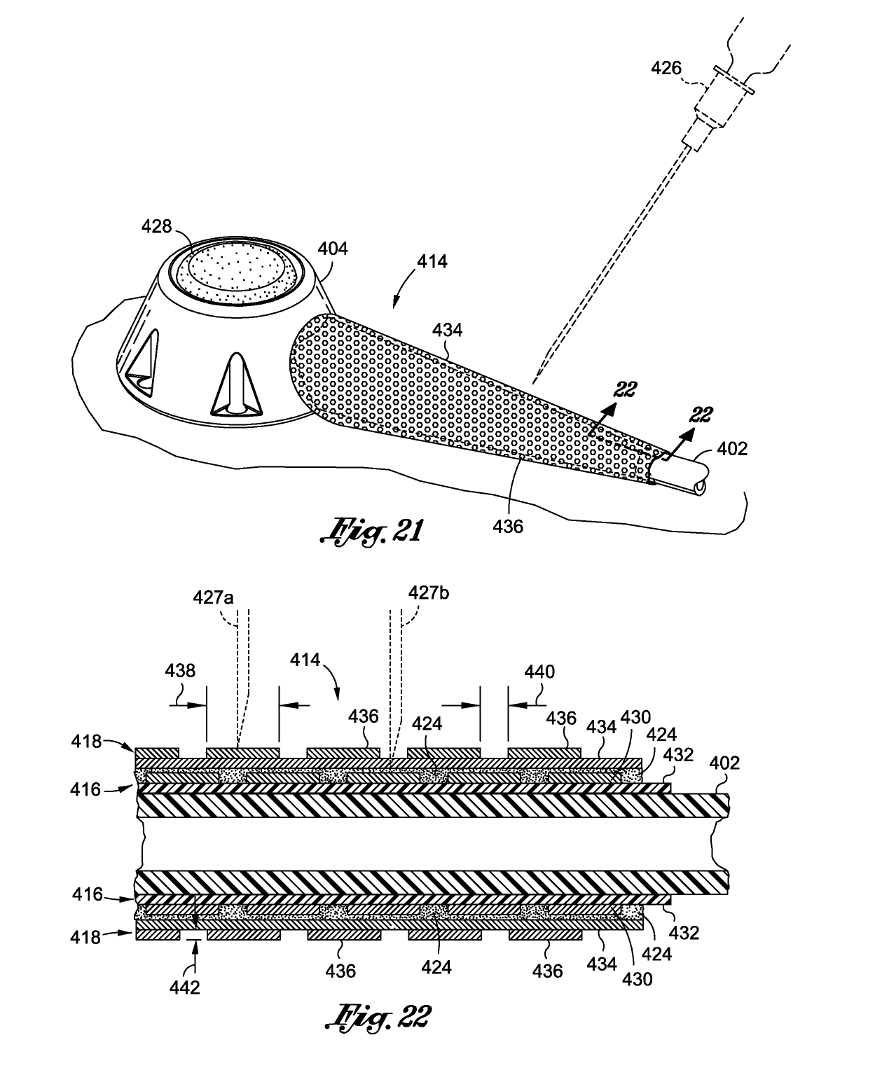

FIG. 21 illustrates a perspective view of the access port 404, the needle guard assembly 414, and the tube 402 shown in FIG. 20. The needle guard assembly 414 forms a sleeve that entirely encircles an outer surface of the end of the tube 402 that connects the access port 404. Thus, the end of the tube 402 connected to the access port 404 is protected in all directions from a syringe 426 needle that has missed the septum 428, and is headed towards the end of the tube 402.

The needle guard assembly 414 has a similar construction as the needle guard assembly 314 discussed in relation to FIGS. 11-19. Namely, the needle guard assembly 414 similarly includes a layered construction of composite guards, connected with an intermediate layer of flexible, connecting material, which bonds the composite guards together. Each composite guard includes a plurality of, for example, an arrangement, array, or pattern of, puncture resistant members, and a flexible substrate having a first side on which the puncture resistant members are positioned. The puncture resistant members are made of a material resistant to puncture by a syringe needle. The flexible substrates are made of a flexible material that provides a degree of compliance for the needle guard assembly 414. In FIG. 21, the puncture resistant members 436 and the flexible substrate 434 of an outer layer of the needle guard assembly 414 are visible.

FIG. 22 illustrates a cross-sectional view of a portion of the tube 402 and the needle guard assembly 414 shown in FIG. 21. The needle guard assembly 414 is shown to comprise a first composite guard 416 covered by a second composite guard 418. The construction of the first composite guard 416 and the second composite guard 418 is similar to the respective constructions of the first composite guard 316 and the second composite guard 318, as described in relation to FIGS. 11-19. Namely, the puncture resistant members 430 of the first composite guard 416 are positioned on one side of a flexible substrate 432. The puncture resistant members 436 of the second composite guard 418 are positioned on one side of a flexible substrate 434. Each puncture resistant member 430, 436 is coupled to the respective flexible substrate 432, 434. An intermediate layer 424 is positioned between, and connects the first composite guard 416 to the second composite guard 418. The first composite guard 416 is positioned between the second composite guard 418 and the tube 402. The second composite guard 418 covers the first composite guard 416.

In the embodiment shown in FIG. 22, the first composite guard 416, the second composite guard 418, and the intermediate layer 424, are shaped to wrap around the entirety of the outer surface of the tube 402. The composite guards 416, 418 each are wrapped to have a substantially cylindrical shape, or conical shape, that allows them to extend entirely around the outer surface of the tube 402.

Similar to the positioning of the composite guards 316, 318, 320, discussed in relation to FIGS. 11-19, the first composite guard 416 and the second composite guard 418 are positioned such that the arrangement of the puncture resistant members 436 of the second composite guard 418 is misaligned with the arrangement of the puncture resistant members 430 of the first composite guard 416. Thus, the composite guards 416, 418 are arranged relative to one another such that there are no straight line open spaces, or substantial gaps, between members 430, 436 to allow a needle or sharp implement to penetrate entirely through the needle guard assembly 414. The puncture resistant members 436 of the second composite guard 418 cover each space between the puncture resistant members 430 of the first composite guard 416. For example, FIG. 22 illustrates a syringe needle 427a impacting a puncture resistant member 436 of the second composite guard 418. However, the composite guards 416, 418 are misaligned such that if an incident syringe needle misses the puncture resistant members 436 of the second composite guard 418, then the syringe needle will contact the puncture resistant members 430 of the first composite guard 416. For example, FIG. 22 illustrates a syringe needle 427b passing through a space between the members 436 of the second composite guard 418, and passing through the flexible substrate 434 of the second composite guard 418, and through the intermediate layer 424. Yet, the syringe needle 427b contacts the puncture resistant member 430 of the first composite guard 416, and does not penetrate the tube 402.

In addition, similar to the embodiments of the needle guard assembly 314 discussed in relation to FIGS. 11-19, the members 436, 430 may each have a thickness 442 of between about 0.1 millimeter (mm) and about 1.0 mm. The members 436, 430 may each have a diameter 438 of between about 0.5 mm and about 2.0 mm, for example, a diameter of about 1.5 mm.

A space may be positioned between adjacent puncture resistant members 436, 430. The space may have a width 440 of between about 0.2 mm and about 0.5 mm.

The puncture resistant members 430, 436, similar to the members 330 discussed in relation to FIGS. 11-19, may be any suitable shape. In FIG. 22, the members 430, 436 are shown to have a round shape, with a flattened, or planar shaped top, similar to the embodiment of the puncture resistant members 330a, shown in FIG. 16. In other embodiments, the members 430, 436 may have any shape shown in FIGS. 15-16a, or as discussed elsewhere in this disclosure. In particular, the members 430, 436 may have a dome shape as shown in FIG. 15, or a dome shape with a flattened, or planar, upper surface, as illustrated in FIG. 16a. In addition, similar to the needle guard assembly 316 discussed in relation to FIGS. 11-19, the needle guard assembly 414 may include between about 50 members and about 1000 members 430, 436 per square inch (psi), for example, about 400 psi.

In one specific embodiment, each composite guard 416, 418 may include about 400 members psi, each having a diameter of about 1.5 mm and each being spaced apart about 0.2 mm.

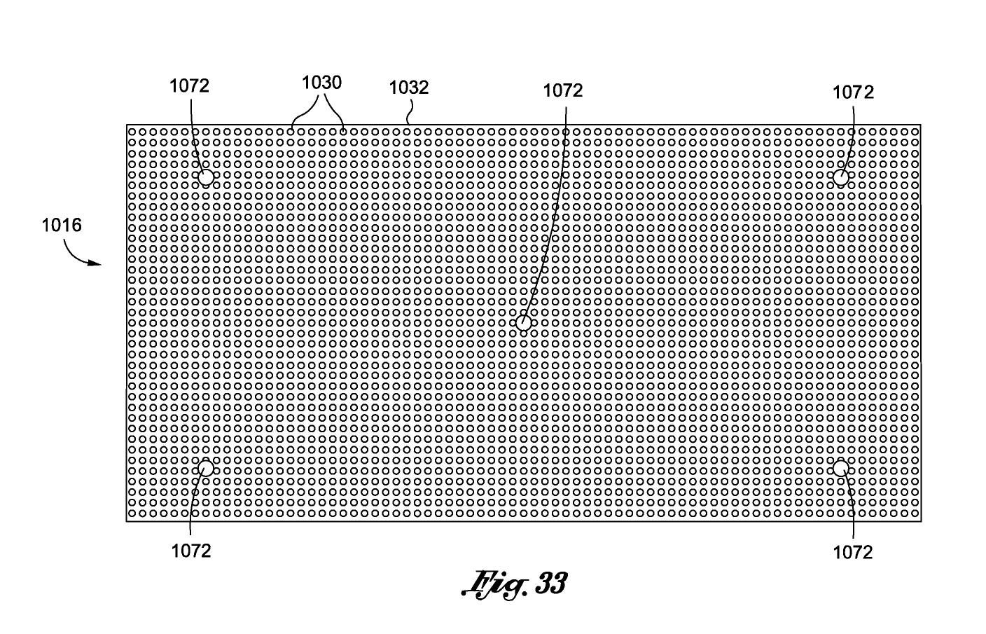

The needle guard assembly 414 may comprise a pre-formed sleeve that is slid over the end of the tube 402 connected to the access port 404. The needle guard assembly 414 may then be glued to the end of the tube 402, through an appropriate adhesive, for example, a silicone-based glue, or the like. In one embodiment, the leading end of the needle guard assembly 414 may be glued directly to the access port 404. In one embodiment, the needle guard assembly 414 may be formed directly on the tube 402. For example, the first composite guard 416 may be cut to the appropriate shape out of a sheet of material, shown for example, in FIG. 31 or 32. The first composite guard 416 may be wrapped around the outer surface of the tube 402 and glued in place. Then, the intermediate layer 424 may be placed over the first composite guard 416. The second composite guard 418 may be cut to size out of a similar sheet of material as the first composite guard, and then fixed to the intermediate layer 424.

The needle guard assembly 414 may be shaped to contour to the shape of the underlying tube 402. For example, FIG. 21 illustrates the needle guard assembly 414 having a conical shape, as it conforms to the underlying conical shape of the tube 402. In other embodiments, the needle guard assembly 414 may have any shape that produces equivalent operation, including a cylindrical shape, or a pyramidal shape, or the like.

The needle guard assembly 414 is positioned on the tube 402 such that no gap exists between the access port housing and the assembly 414, such that a needle could not penetrate the portion of the tube 402 that is directly connected to the access port 404. The needle guard assembly 414 thus completely protects the end of the tube 402 connected to the access port 404. In other embodiments, the needle guard assembly 414 may only cover a portion of the tube 402, or may not wrap entirely around the outer surface of the tube 402. For example, the needle guard assembly 414 may be configured to only cover the uppermost portion of the tube 402, or the portion facing the nearest surface of the patient's skin. The amount of protection offered by the needle guard assembly 414 may be varied as desired.

In the embodiment of the needle guard assembly 414 shown in FIGS. 21 and 22, the outer surface of the needle guard assembly 414 has a bumpy surface, caused by the spacing between the puncture resistant members 436 of the second composite guard 418. In other embodiments, the needle guard assembly 414 may be covered by a material, for example, an elastomeric material, to smooth the surface of the needle guard assembly 414.

The layered composition of the needle guard assembly 414 may be varied as desired. For example, the number of composite guards utilized with the needle guard assembly 414 may be varied, from two guards to four guards. In other embodiments, additional guards may be utilized, if equivalent operation results. In one embodiment, only one composite guard may be utilized, with puncture resistant members spaced closely to each other, or close enough to block an incoming syringe needle. In addition, in other embodiments, the orientation of the composite guards may additionally be varied. For example, in one embodiment, the second composite guard 418 may be flipped such that the puncture resistant members 436 of the second composite guard 418 face the puncture resistant members 430 of the first composite guard 416. In other words, the puncture resistant members 436 of the second composite guard 418 may be positioned on the other side of the flexible substrate 434, than shown in FIG. 22.

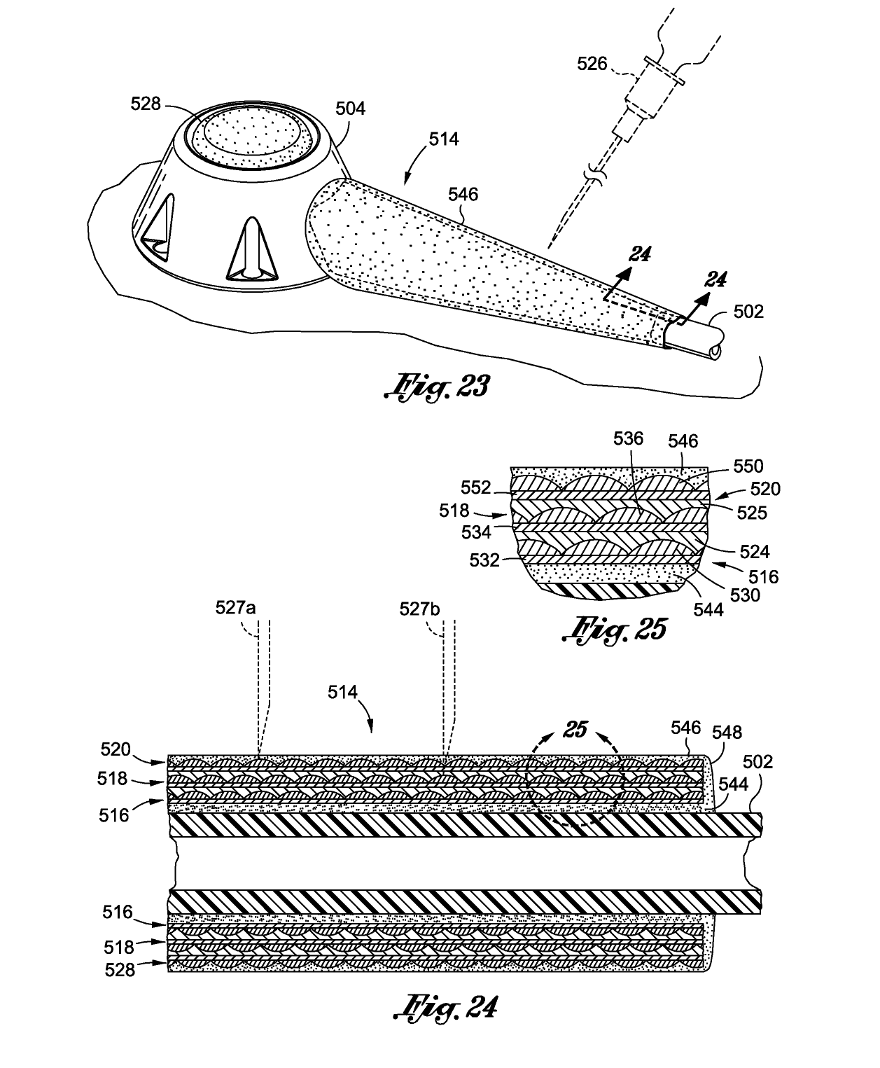

FIG. 23 illustrates an embodiment of a needle guard assembly 514, similar to the needle guard assembly 414 discussed in relation to FIGS. 20-22, which is configured to protect an end of a tube 502 connected to an access port 504. The needle guard assembly 514 similarly blocks the passage of a syringe 526 needle that has missed contacting the septum 528 of the access port 504. However, the layered composition of the needle guard assembly 514 differs from the composition of the needle guard assembly 414 discussed in relation to FIGS. 20-22. The needle guard assembly 514 shown in FIG. 23 includes three composite guards, two intermediate layers, a bottom layer, and a top layer 546 that is visible in FIG. 23.

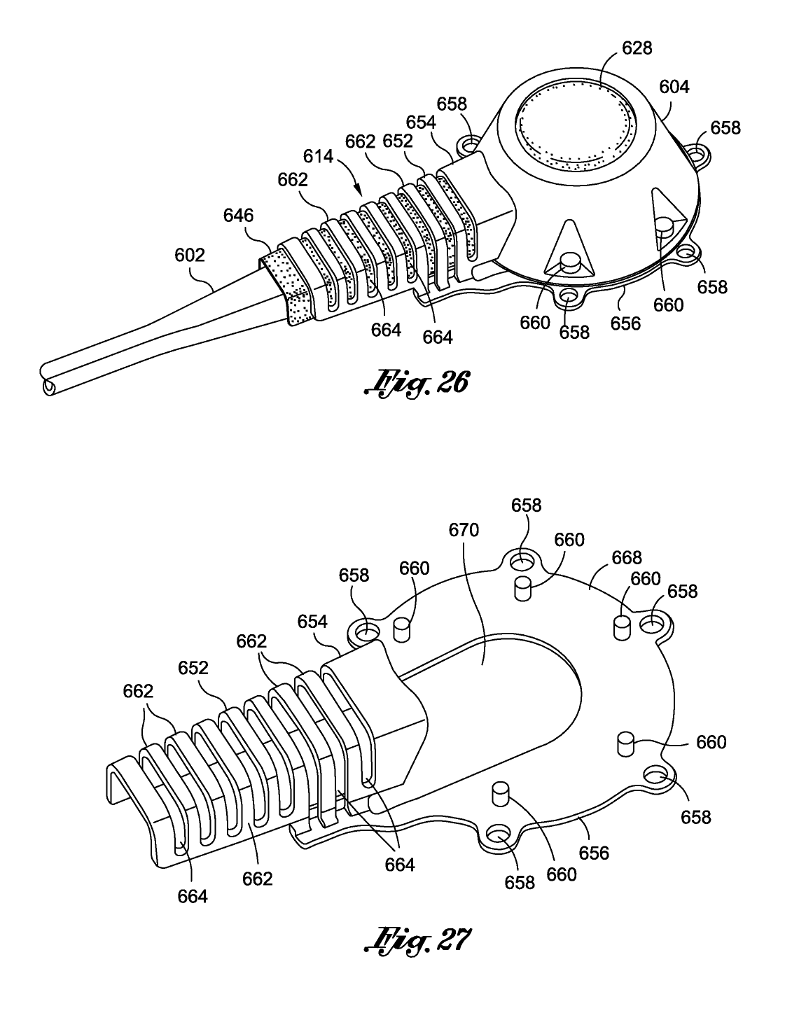

FIG. 24 illustrates a cross sectional view of the needle guard assembly 514, and the tube 502 shown in FIG. 23. FIG. 25 shows a close up view of a portion of the needle guard assembly 514 as shown in FIG. 24. Referring to both FIGS. 24 and 25, the needle guard assembly 514 includes a first composite guard 516, a second composite guard 518, and a third composite guard 520. A first intermediate layer 524 is positioned between the first composite guard 516, and the second composite guard 518. A second intermediate layer 525 is positioned between the second composite guard 518 and the third composite guard 520. Each composite guard 516, 518, 520 has a similar composition as the guards forming the needle guard assemblies 314, 414, discussed in relation to FIGS. 11-22. Namely, each composite guard 516, 518, 520 includes an arrangement, array, or pattern of, respective puncture resistant members 530, 536, 550, and a respective flexible substrate 532, 534, 552 having a first side on which the respective puncture resistant members 530, 536, 550, are positioned, or coupled thereto. The first and second intermediate layers 524, 525 have similar compositions as the intermediate layers 324, 424 discussed in relations to FIGS. 11-22.

The composite guards 516, 518, 520, each include respective puncture resistant members 530, 536, 550, that do not have a space positioned between adjacent puncture resistant members. As shown in FIG. 25, each of the puncture resistant members 530, 536, 550, directly contacts an adjacent puncture resistant member. Yet, each composite guard 516, 518, 520, is still misaligned with at least one other composite guard (e.g., the third composite guard 520 is misaligned with at least one of the first composite guard 516, or the second composite guard 518), to assure that no straight line open spaces, or substantial gaps, between members 530, 536, 550, may exist to allow a needle or sharp implement to penetrate entirely through the assembly 514, and contact the tube 502. The lack of space between adjacent puncture resistant members 530, 536, 550, further enhances the ability of the needle guard assembly 514 to prevent needle penetration. For example, FIG. 24 illustrates syringe needles 527a, 527b, unable to penetrate even the outermost composite guard 520.

The needle guard assembly 514 includes an inner or bottom layer 544 that is positioned below the first composite guard 516. The needle guard assembly 514 includes an outer or top layer 546 that is positioned above the third composite guard 520. The top layer 546 and bottom layer 544 each comprise a soft elastomeric material, such as silicone. The top layer 546 and the bottom layer 544 may connect at an end 548 of the layers 546, 544, to enclose the composite guards 516, 518, 520, and the intermediate layers 524, 525, and to provide a fluid tight barrier for the interior of the needle guard assembly 514.

Aside from the additional top layer 546, the bottom layer 544, the second intermediate layer 525, the third composite guard 520 and the spacing of the puncture resistant members 530, 536, 550, the needle guard assembly 514 includes similar construction as the needle guard assembly 414 discussed in relation to FIGS. 20-22. Namely, the thickness and the diameter of the puncture resistant members 530, 536, 550, may be the same as the members of the needle guard assembly 414 discussed in relation to FIGS. 20-22. In addition, the shape and the amount of the puncture resistant members 530, 536, 550 on the needle guard assembly 514, may be the same as for the members of the needle guard assembly 414 discussed in relation to FIGS. 20-22. In addition, the sleeve shape of the needle guard assembly 514, and the position of the needle guard assembly 514 along the tube 502, may be the same as for the needle guard assembly 414 discussed in relation to FIGS. 20-22.

Although the needle guard assembly 514 is shown without any space between adjacent puncture resistant members 530, 536, 550, in one embodiment, the puncture resistant members 530, 536, 550, may include spaces, and the spaces may be sized the same as for the members of the needle guard assembly 414 discussed in relation to FIGS. 20-22. In addition, the construction of the needle guard assembly 514 as a pre-formed sleeve, or as a series of layers formed directly on the tube 502, may be identical to the construction of the needle guard assembly 414 discussed in relation to FIGS. 20-22. The three-composite layers preferably enhance the protection offered by the needle guard assembly 514. The three composite guard layers provide increased protection for the tube 502 from needle puncture compare to the two composite guards and/or the needle guard assembly 414 discussed in relation to FIGS. 20-22.

The needle guard assemblies 414, 514, discussed in relation to FIGS. 20-25, beneficially provide a flexible protective material over an end of the respective tubes 402, 502. The needle guard assemblies 414, 514 may each also have a resiliency or a shape memory such that it will restore it to a particular shape after being manipulated by a physician. This may be provided by using a suitable intermediate layer material, such as a silicone elastomer that has a shape memory characteristic. The flexibility of each of the needle guard assemblies 414, 514 may reduce discomfort for the patient upon insertion of the respective access ports 404, 504. In addition, the flexibility of each of the needle guard assemblies 414, 514, may allow a physician to more easily position the respective access port 404, 504, and the tube 402, 502, within a patient's body during implantation.

The needle guard assemblies 414, 514, discussed in relation to FIGS. 20-25, may be utilized with access ports that are not part of the gastric banding systems. Although a gastric banding system is one intended embodiment of the present invention, the needle guard assemblies 414, 514 may be used to protect tubing attached to any implantable access port, including, but not limited to, a drug eluting access port, an access port used to control the pressure of a urinary restriction device, or anal incontinence device, or the like. The needle guard assemblies 414, 514 may be used in any implantable medical device utilizing an access port, and having a component that requires protection from puncture.

The puncture resistant members 430, 436, 530, 536, 550 shown in FIGS. 20-25 may be made of a similar puncture resistant material as the puncture resistant members 330 (and 330a and 330b) discussed in relation to FIGS. 11-19. Namely, the members 430, 436, 530, 536, 550 are made of a suitable puncture resistant material, such as an epoxy, acrylic materials, hot-melt adhesives, thermoplastics, polymer, rubber, ceramic or metal, or suitable combination or alloy thereof. For some applications, suitable materials include polyethylene (PE), polypropylene (PP), polyurethane (PU), polyethylene terephthalate (PET), polycarbonate (PC), polyisoprene (PI), thermoplastic urethanes and thermoplastic polyurethanes (TPU), high durometer silicones, acrylonitrile butadiene styrene (ABS) etc. In some embodiments, the members 430, 436, 530, 536, 550 may be made from a UV-curable epoxy. In some embodiments, the members 430, 436, 530, 536, 550 are made of a material such as acetal, nylon, polycarbonate, and combinations thereof. In some embodiments, the members 430, 436, 530, 536, 550 are made of a metal, for example, stainless steel, aluminum, titanium, or other metal.

The flexible substrates 432, 434, 532, 534, 552 shown in FIGS. 20-25 may be made of a similar flexible material as the flexible substrate 332 discussed in relation to FIGS. 11-19. Namely, the flexible substrates 432, 434, 532, 534, 552 may comprise a mesh, film, fabric, elastomer, or other suitable material. The flexible substrates 432, 434, 532, 534, 552 may be made from thin polyimide, polyester, or other biocompatible film with appropriate thickness. The flexible substrates 432, 434, 532, 534, 552 may be made from silicone, polyurethane, or other foam materials. The flexible substrates 432, 434, 532, 534, 552 may be made from woven or non-woven mesh materials such as Nylon or Polyester. In one embodiment, the flexible substrates 432, 434, 532, 534, 552 may be made from Kapton film. In one embodiment, the flexible substrates 432, 434, 532, 534, 552 may be made from a polymer foam or plastic film.

The intermediate layers 424, 524, 525 shown in FIGS. 20-25 may be made of a similar material as the intermediate layers 324 discussed in relation to FIGS. 11-19. Namely, the intermediate layers 424, 524, 525 may be a polymer, for example, an elastomeric material, such as an elastomeric polymer, for example, a silicone elastomer, for example, a low durometer silicone rubber. The intermediate layers 424, 524, 525 may comprise a soft, tacky layer of elastomeric material, generally comprised of silicone. The top layer 546 and the bottom layer 544 shown in FIGS. 24 and 25 may be made of similar materials discussed above for the intermediate layers 424, 524, 525.

All materials used to form the needle guard assemblies 414, 514 may be biocompatibility rated at USP Class VI. In addition, an encapsulating layer of an elastomeric material, forming a top layer 546 and a bottom layer 544 may be a grade of silicone designed for long term implantation (e.g., 5 or more years of implantation).

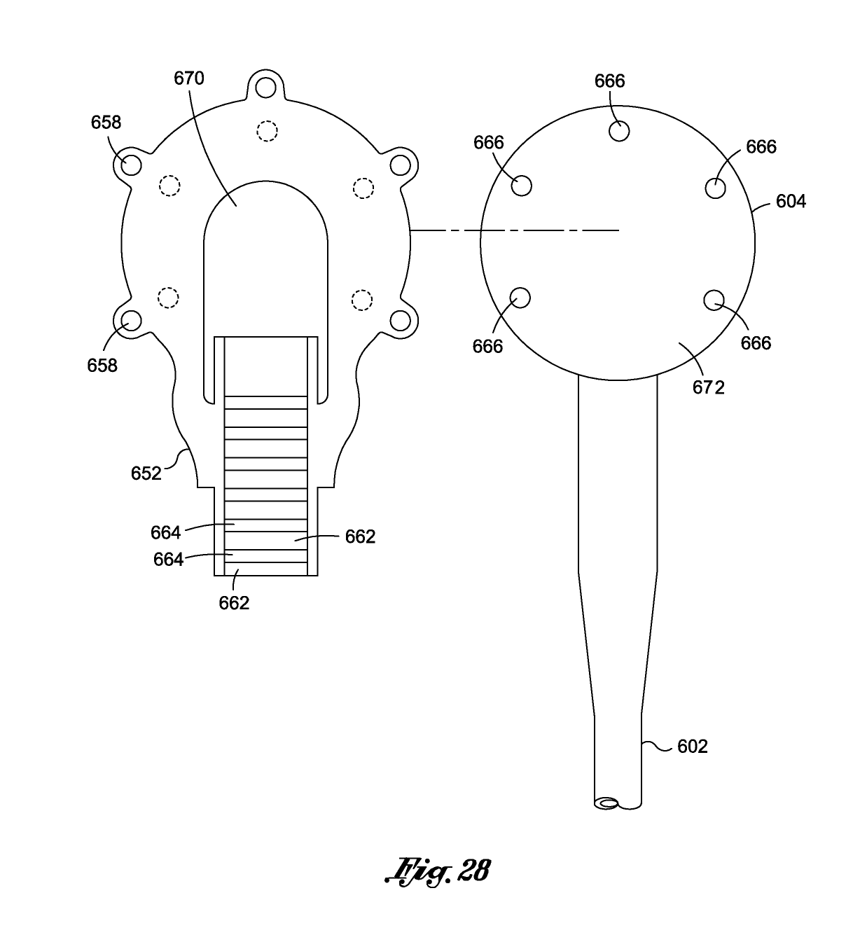

FIG. 26 illustrates a needle guard assembly 614 utilized in conjunction with a clip 652. The needle guard assembly 614 may be configured similarly as the needle guard assembly 514 shown and discussed in relation to FIGS. 23-25. In particular, the needle guard assembly 614 may comprise a layered assembly of composite guards and intermediate layers. The needle guard assembly 614 may include a first composite guard, a second composite guard, and a third composite guard that each have similar compositions as the respective first composite guard 516, the second composite guard 518, and the third composite guard 520 shown in FIGS. 24 and 25. Namely, the first composite guard, the second composite guard, and the third composite guard of the needle guard assembly 614 may include an arrangement, array, or pattern of, respective puncture resistant members, and a respective flexible substrate having a first side on which the respective puncture resistant members are positioned, or coupled thereto.

The needle guard assembly 614, similarly as the needle guard assembly 514 discussed in relation to FIGS. 23-25, may also include a top layer 646, a bottom layer, an intermediate layer between a first composite guard and a second composite guard, and an intermediate layer between a second composite guard and a third composite guard. The layers of the needle guard assembly 614 may have similar compositions as the respective top layer 546, bottom layer 544, and intermediate layers 524, 525 shown and described in relation to FIGS. 23-25. The outer surface of the top layer 646 is visible in FIG. 26.

The needle guard assembly 614 shown in FIG. 26 forms a sleeve that extends partially around the outer surface of the tube 602. The needle guard assembly 614 covers the upper, or top surface of the tube 602, and the sides of the tube 602.

The clip 652 includes a sleeve portion 654 and a mounting portion 656. The sleeve portion 654 is a portion of the clip 652 that extends over, and overlays, the needle guard assembly 614. The sleeve portion 654 secures the needle guard assembly 614 to the end of the tube 602 connected to the access port 604. In the embodiment shown in FIG. 26, the sleeve portion includes a plurality of ribs 662 separated by slots 664. Each rib 662 and each slot 664 extends transverse to the length of the tube 602. The ribs 662 of the sleeve portion 654 overlay the needle guard assembly 614. The slots 664 separate the ribs 662 and provide flexibility for the sleeve portion 654.

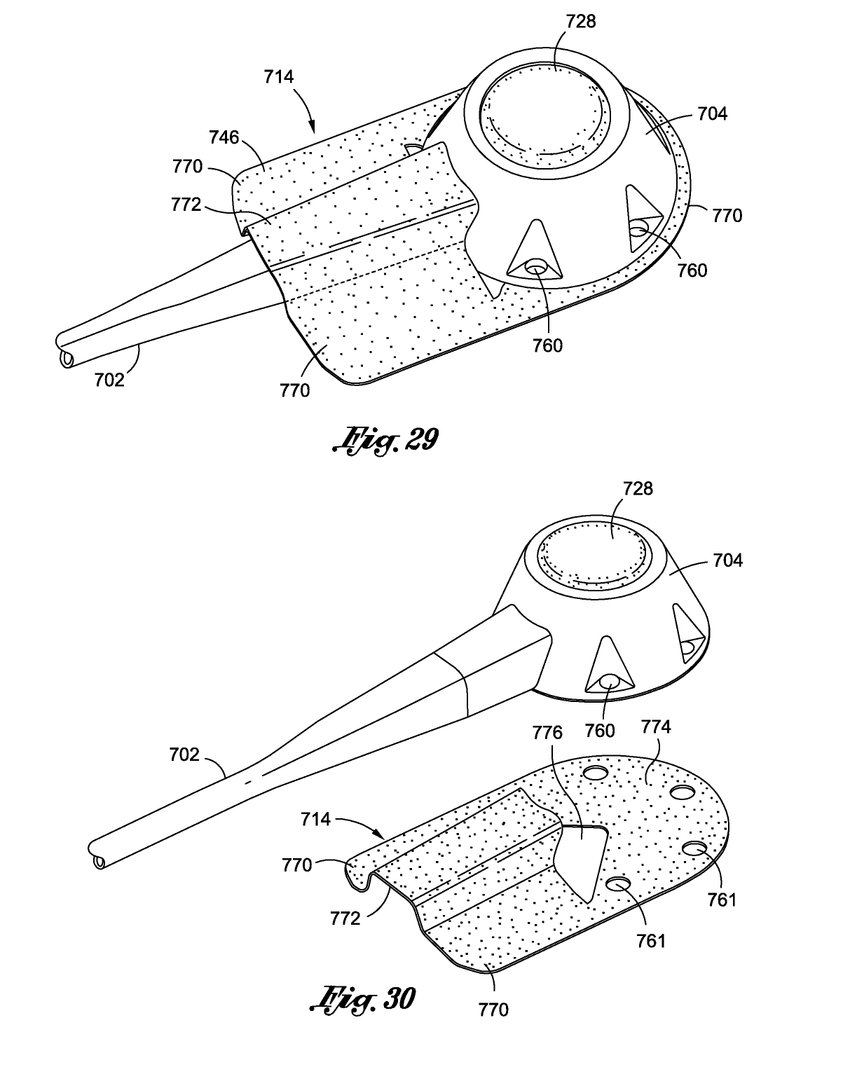

The mounting portion 656 of the clip 652 couples the sleeve portion 654 to the access port 604. In the embodiment shown in FIG. 26, the mounting portion 656 comprises a substantially flattened, or planar portion, of the clip 652 that extends beneath the access port 604, to mount to suture holes 666 (shown in FIG. 28) in the access port 604. The mounting portion 656 includes a plurality of posts 660 that extend vertically from an upper surface of the mounting portion 656, to pass through the access port 604 suture holes. The posts 660 secure the clip 652 to the access port 604 via a friction fit between the posts 660 and the access port 604 suture holes. The clip 652 may additionally include suture holes 658 that extend outward or radially from the mounting portion 656 of the clip 652. The suture holes 658 are configured to allow a user to secure the access port 604 to a portion of a patient's body, in lieu of the suture holes extending through the access port 604 itself.

FIG. 27 illustrates a perspective view of the clip 652 separated from the access port 604. The mounting portion 656 of the clip 652 is shown to comprise a substantially flattened surface 668 configured to abut a bottom surface of the access port 604. In addition, the posts 660 are shown to extend from the surface 668 of the clip 652, and being positioned on the surface 668 to mate with corresponding suture holes 666 of the access port 604, shown in FIG. 28. FIG. 27 additionally illustrates an aperture 670 positioned in the center of the mounting portion 656. The mounting portion 656 forms a flattened ring around the aperture 670.

FIG. 28 illustrates a bottom view of the clip 652 separated from the access port 604. The access port 604 includes a substantially flat bottom surface 672 that abuts the substantially flattened surface 668 of the clip 652, shown in FIG. 27.

Referring to FIGS. 26-28, the clip 652 may be removably secured to the access port 604. The posts 660 of the clip 652 may be slidably removable from the suture holes 666 of the access port 604, such that the entire clip 652 may be removed from the access port 604. To install the clip 652 onto the access port 604 from an initially separated configuration, the tube 602 may be initially passed through the aperture 670 of the clip 652. Then, once the clip 652 nears the access port 604, the clip 652 may be rotated such that the posts 660 pass through the suture holes 666 of the access port 604. As the posts 660 enter the suture holes 666, the tube 602 will enter the cavity formed by the sleeve portion 654 of the clip 652. The posts 660 secure the clip 652 against the access port 604. The clip 652 secures the needle guard assembly 614 to the tube 602. To remove the clip 652 from the access port 604, the installation process is reversed.

The clip 652 beneficially serves to secure the needle guard assembly 614 against the end of the tube 602 that is coupled to the access port 604. The clip 652 presses the needle guard assembly 614 against the outer surface of the tube 602 to secure the assembly 614 in position on the tube 602. Thus, the needle guard assembly 614 does not need to be directly adhered to the tube 602, or form a sleeve extending entirely around the outer surface of the tube 602. The clip 652 may press-fit the needle guard assembly 614 in place against the tube 602.

The sleeve portion 654 of the clip 652 is preferably made flexible to accommodate movement of the needle guard assembly 614. The slots 664 separating the ribs 662 of the sleeve portion 654 provide flexibility for the sleeve portion 654 by removing material between the ribs 662. In addition, the slots 664 reduce the total weight of the clip 652.

The clip 652 additionally serves as an additional protective layer over the needle guard assembly 614. The clip 652 may be made of a needle-impenetrable material such as plastic. If an incident needle misses the septum 628 of the access port 604 and contacts the clip 652, then the clip 652 may block movement of the incident needle, and prevent the needle from puncturing the tube 602.

The clip 652 may be configured in a variety of shapes designed to equivalently secure the needle guard assembly 614 in position on the tube 602. In one embodiment, the sleeve portion 654 of the clip 652 may be a substantially solid sleeve that does not include ribs 662 or slots 664. In one embodiment, the sleeve portion 654 may include slots 664 that extend lengthwise along the tube 602. In one embodiment, the mounting portion 656 of the clip 652 may extend over a top surface of the access port 604 or wrap around an outer surface of the access port 604. In one embodiment, the clip 652 may be configured as any device capable of securing a needle guard assembly in position on tubing.

The clip 652 is preferably made of a needle-impenetrable, yet flexible material such as plastic. Other equivalent materials, capable of securing the needle guard assembly 614 in position to the tube 602 may also be utilized.

In one embodiment, the clip 652 is adhered to the access port 604. In this embodiment, the posts 660 may be adhered to the suture holes 666 of the access port 604 with a suitable biocompatible adhesive. In addition, in one embodiment, a suitable biocompatible adhesive may be positioned on the upper surface 668 of the clip 652, to adhere the clip 652 to a bottom surface 672 of the access port 604.

In one embodiment, the needle guard assembly 614 may be directly adhered to the clip 652.

FIG. 29 illustrates a needle guard assembly 714 having a flanged portion 770 extending from a sleeve portion 772 of the needle guard assembly 714. The needle guard assembly 714 may be configured similarly as the needle guard assembly 514 shown and discussed in relation to FIGS. 23-25. In particular, the needle guard assembly 714 may comprise a layered assembly of composite guards and intermediate layers. The needle guard assembly 714 may include a first composite guard, a second composite guard, and a third composite guard that each have similar compositions as the respective first composite guard 516, the second composite guard 518, and the third composite guard 520 shown in FIGS. 24 and 25. Namely, the first composite guard, the second composite guard, and the third composite guard of the needle guard assembly 714 may include an arrangement, array, or pattern of, respective puncture resistant members, and a respective flexible substrate having a first side on which the respective puncture resistant members are positioned, or coupled thereto.

The needle guard assembly 714, similarly as the needle guard assembly 514 discussed in relation to FIGS. 23-25, may also include a top layer 746, a bottom layer, an intermediate layer between a first composite guard and a second composite guard, and an intermediate layer between a second composite guard and a third composite guard. The layers of the needle guard assembly 714 may have similar compositions as the respective top layer 546, bottom layer 544, and intermediate layers 524, 525 shown and described in relation to FIGS. 23-25. The outer surface of the top layer 746 is visible in FIG. 29.

The sleeve portion 772 of the needle guard assembly 714 is shaped to substantially contour to the shape of the tube 702. In the embodiment shown in FIG. 29, the sleeve portion 772 does not extend entirely around the outer surface of the tube 702, but only extends over the top of the tube 702 and the sides of the tube 702. A flanged portion 770 of the needle guard assembly 714 extends outward from the sleeve portion 772. A part of the flanged portion 770 may extend beneath the access port 704, to adhere to the bottom of the access port 704.

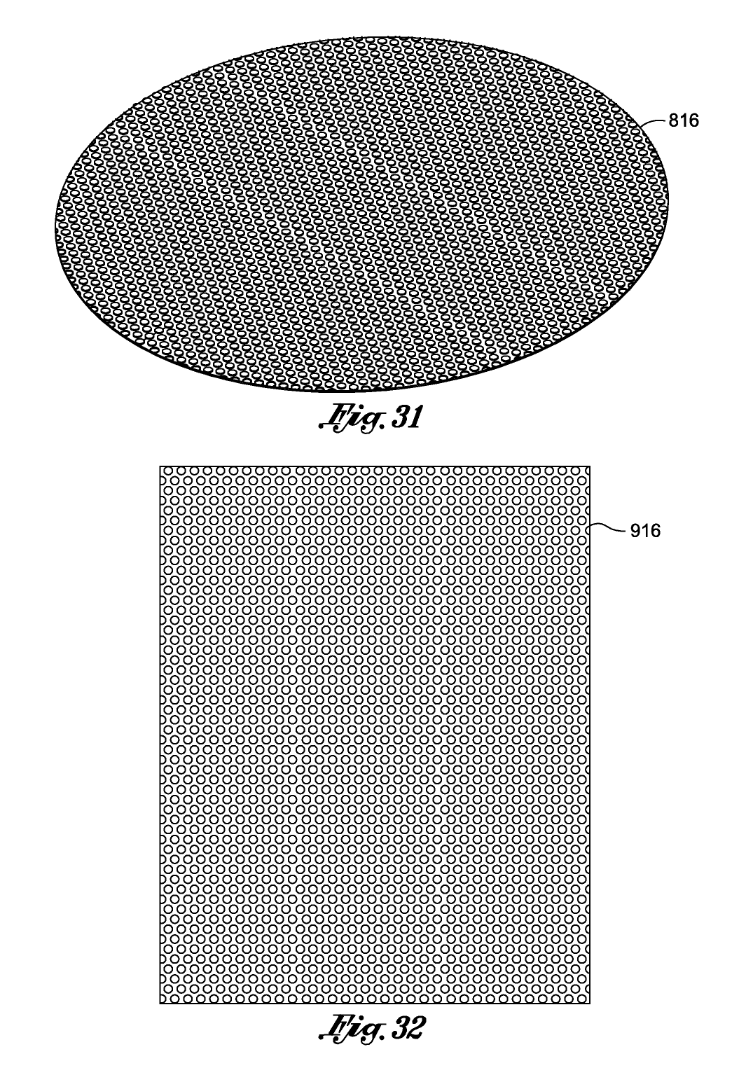

The access port 704 may be fixed to a portion of the patient's body by use of the suture holes 760 on the access port 704. The needle guard assembly 714 may include suture holes (shown in FIG. 30) that correspond to the location of the access port's 704 suture holes 760.

FIG. 30 illustrates the needle guard assembly 714 separated from the access port 704. The upper surface 774 of the needle guard assembly 714, which fixes to the access port 704, is visible. In addition, an aperture 776 in the needle guard assembly 714 is visible. The suture holes 761 in the flanged portion 770 of the needle guard assembly 714, which correspond to the suture holes 760 of the access port 704, are visible.

To secure the needle guard assembly 714 to the access port 704 from an initially separated configuration, as shown in FIG. 30, the tube 702 may be initially passed through the aperture 776 of the needle guard assembly 714. Then, once the needle guard assembly 714 nears the access port 704, the upper surface 774 of the needle guard assembly 714 may be adhered to the access port 704. The adhesive may be a soft, biocompatible silicone rubber material, such as Nusil MED-4805, or 4810, or the like. The adhesive may be applied to the entire surface of the needle guard assembly 714 and may act as a bonding agent to the access port 704 while curing.

The sleeve portion 772 of the needle guard assembly 714 is positioned above the tube 702, to prevent incident needles from puncturing the tube 702. The flanged portion 770 of the needle guard assembly 714 is positioned above a portion of the patient's body tissue, to prevent incident needle from penetrating the patient's body tissue. As discussed above, the access port 704 is preferably fixed to tissue such as a patient's muscle wall. If an incident needle missed the septum 728 and penetrated the patient's muscle wall, the patient would likely experience great pain. The flanged portion 770 of the needle guard assembly 714 protects the patient's muscle wall from incident needle penetration. The flanged portion 770 additionally serves to strengthen the needle guard assembly 714, and to serve as an attachment structure for the needle guard assembly 714 to the access port 704.

The needle guard assembly 714 may be configured in a variety of shapes designed to equivalently protect the tube 702 from puncture. In one embodiment, the flanged portion 770 may have a curved shape, or any variety of equivalent shapes extending from a sleeve portion 772 of the needle guard assembly 714. In one embodiment, the flanged portion 770 may extend over a top surface of the access port 704 or wrap around an outer surface of the access port 704. In one embodiment, the needle guard assembly 714 may have any equivalent shape capable of protecting the tube 702 from puncture.

The composite guards 416, 418, 516, 518, 520 shown in FIGS. 20-25, or discussed in relation to FIGS. 26-30, may be formed in a similar manner as the guards 316 discussed in relation to FIGS. 11-19. For example, the composite guards 416, 418, 516, 518, 520 may be formed by a suitable method, including stencil printing, for example, using equipment and processes used in surface mount technology/PCB fabrication. Other processes that can be used to make the composite guards 416, 418, 516, 518, 520 include micro-dot dispensing and printing, laser etching, and stencil printing. For example, a uniform film of a hard encapsulant may be applied on the flexible substrate and etched with laser etching/engraving equipment to achieve a desired pattern. Other suitable methods will be known to those of skill in the art. Referring to FIGS. 31 and 32, the composite guards 816, 916 are initially formed in the shape of generally flat sheets. The formation methods result in composite guards 816, 916 with puncture resistant members bonded to the flexible substrates. A composite guard 416, shown in FIG. 22, for example, will then be cut from the sheets to the appropriate shape, as desired.

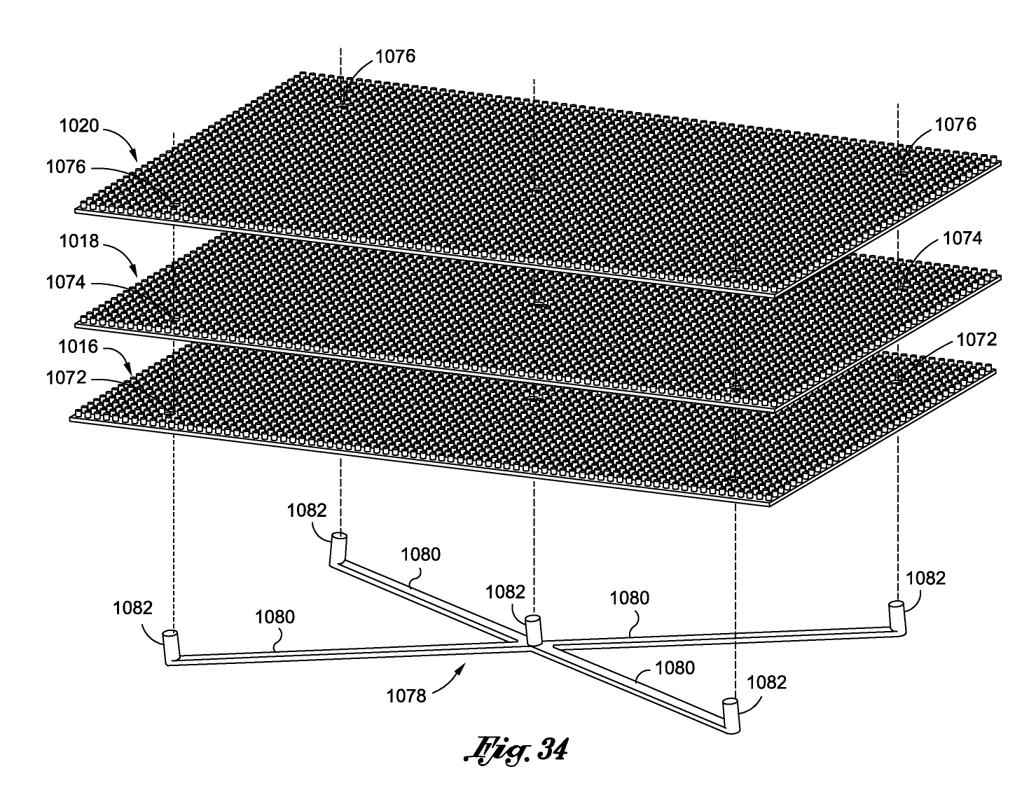

In addition, the intermediate layers 424, 524, 525 shown in FIGS. 20-25, or discussed in relation to FIGS. 26-30 may be formed in a similar manner as the intermediate layers 324 discussed in relation to FIGS. 11-19, and as specifically shown in FIGS. 17-19. For example, a suitable material for the intermediate layer 424, 524, 525, for example, a sheet of uncured silicone, is placed on one side of the respective composite guard 416, 418, 516, 518, 520, for example, on the side having the respective puncture resistant members 430, 436, 530, 536, 550. The sheet is then subjected to curing conditions to cause the sheet to adhere to the respective puncture resistant members 430, 436, 530, 536, 550, forming respective intermediate layer 424, 524, 525 thereon. Such curing conditions may include placement of the sheet in an oven. In an embodiment in which the needle guard assemblies 414, 514 are formed directly on the respective tubes 402, 502, the composite guards 416, 418, 516, 518, 520 and the intermediate layers 424, 524, 525 may be placed on the respective tubes 402, 502 prior to the curing step. In an embodiment including a bottom layer 544 and/or a top layer 546, the bottom layer 544 and/or the top layer 546 may be appropriately positioned on the tube 502 prior to curing. The methods of forming composite guards 816, 916 may additionally be used to form the composite guards discussed in relation to FIGS. 33-38.

FIG. 33 illustrates an embodiment of a composite guard 1016 including registration holes 1072 extending through the surface of the composite guard 1016. The composite guard 1016 may have a similar composition as the respective first composite guard 516 shown in FIGS. 24 and 25. Namely, the composite guard 1016 may include an arrangement, array, or pattern of, respective puncture resistant members 1030, and a respective flexible substrate 1032 having a first side on which the respective puncture resistant members 1030 are positioned, or coupled thereto.

The registration holes 1072 are positioned on the composite guard 1016 in a manner such that the arrangement of puncture resistant members 1030 on the flexible substrate 1032 is misaligned with the arrangement of puncture resistant members of at least one other composite guard. FIG. 34 illustrates an embodiment in which three composite guards are utilized to form a needle guard assembly. The first composite guard 1016 is utilized in combination with a second composite guard 1018, and a third composite guard 1020. The second composite guard 1018 and the third composite guard 1020 may have a similar composition as the respective second composite guard 518, and the third composite guard 520 shown in FIGS. 24 and 25. Namely, the composite guards 1018, 1020 may each include an arrangement, array, or pattern of, respective puncture resistant members, and a respective flexible substrate having a first side on which the respective puncture resistant members are positioned, or coupled thereto.

The second composite guard 1018 and the third composite guard 1020 include respective registration holes 1074, 1076. The position of the registration holes 1072 of the first composite guard 1016 corresponds to the position of the registration holes 1076 of the third composite guard 1020, such that the arrangement of puncture resistant members of the first composite guard 1016 is aligned with the arrangement of puncture resistant members of the third composite guard 1020. The position of the registration holes 1072 of the first composite guard 1016 corresponds to the position of the registration holes 1074 of the second composite guard 1018, such that the arrangement of puncture resistant members of the first composite guard 1016 is misaligned, or offset, with the arrangement of puncture resistant members of the second composite guard 1018. The puncture resistant members of the first composite guard 1016 are misaligned, or offset, with the arrangement of puncture resistant members of the second composite guard 1018 by half the distance between two adjacent puncture resistant members.

A frame 1078 is passed through the registration holes 1072, 1074, 1076 of the respective first composite guard 1016, the second composite guard 1018, and the third composite guard 1020 to maintain the puncture resistant members in position relative to each other during construction of the needle guard assembly. The frame 1078 includes pins 1082 and arms 1080 connecting the pins 1082. The pins 1082 extend through the registration holes 1072, 1074, 1076 to maintain the misalignment between the first composite guard 1016 and the second composite guard 1018. The frame 1078 may be thin, and flexible, and made out of a biocompatible grade plastic, such as PEEK or PolySulfone, or PolyPhenylSulfide, or the like. The frame 1078 may additionally be made of a thin metal such as titanium, or stainless steel, or the like.

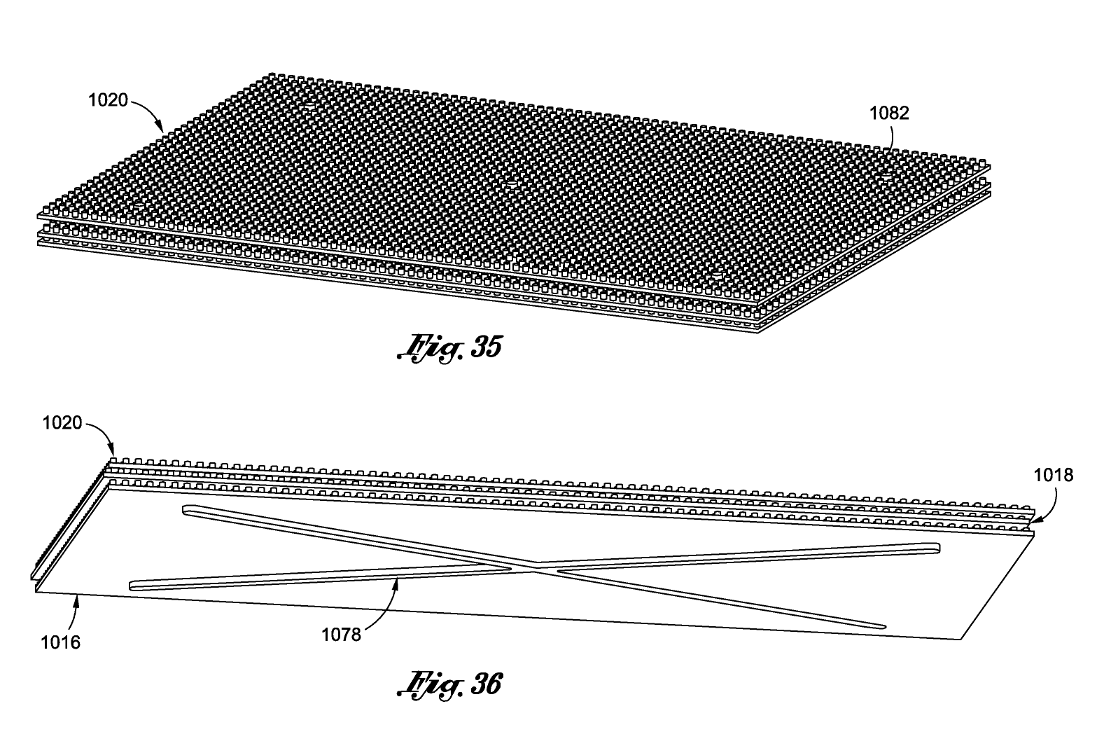

FIGS. 35 and 36 illustrates perspective views of the first composite guard 1016, the second composite guard 1018, and the third composite guard 1020 after the pins 1082 have passed through the respective registration holes 1072, 1074, 1076 (shown in FIG. 34). FIG. 36 illustrates a bottom perspective view of the composite guards 1016, 1018, 1020 with the frame 1078 abutting a bottom surface, or side, of the third composite guard 1016.

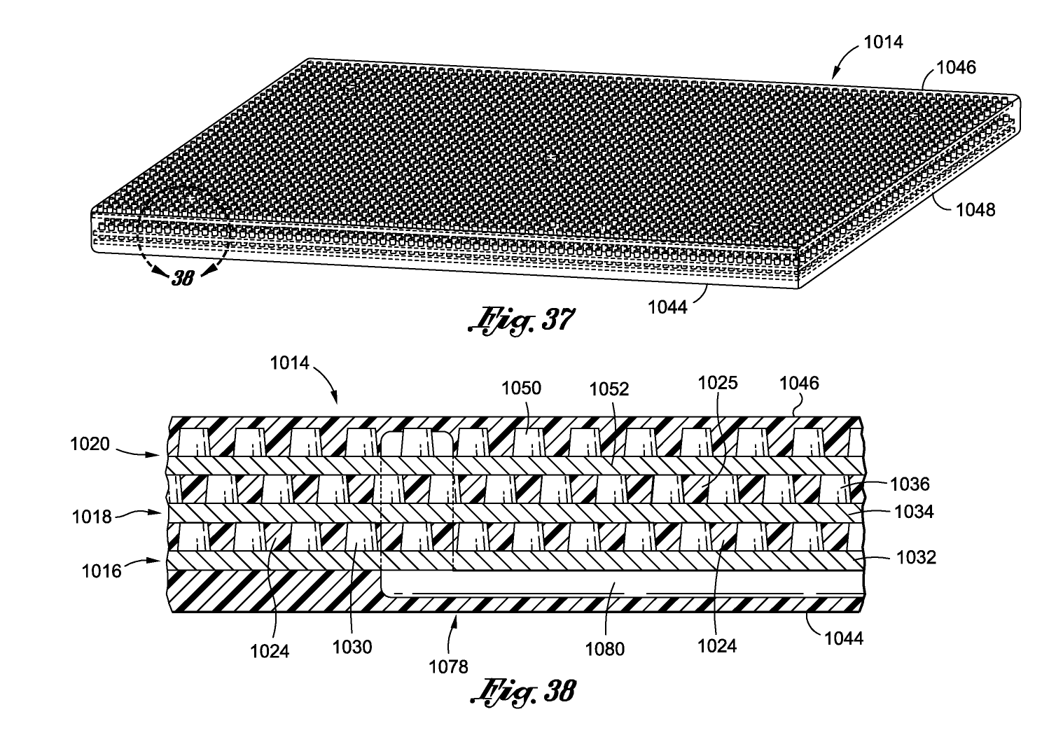

Once the composite guards 1016, 1018, 1020 are in position relative to each other, with the frame 1078 extending through the registration holes 1072, 1074, 1076 (shown in FIG. 34), then an overmolding of an elastomeric material may be formed over the composite guards 1016, 1018, 1020 and the frame 1078. FIG. 37 illustrates a needle guard assembly 1014 formed after the overmolding of an elastomeric material. The overmolding entirely encapsulates the composite guards 1016, 1018, 1020 and the frame 1078, visible in FIG. 38. The overmolding forms a top layer 1046, a bottom layer 1044 and an enclosing side end 1048 of elastomeric material.

FIG. 38 illustrates a detail view of the needle guard assembly 1014 shown in FIG. 37. The puncture resistant members 1030 and the flexible substrate 1032 of the first composite guard 1016 are shown in a layered configuration below the puncture resistant members 1036 and the flexible substrate 1034 of the second composite guard 1018, and the puncture resistant members 1050 and the flexible substrate 1052 of the third composite guard 1020. The frame 1078 extending through the composite guards 1016, 1018, 1020 maintains the misalignment between the first composite guard 1016 and the second composite guard 1018. The frame 1078 additionally maintains the alignment between the first composite guard 1016 and the third composite guard 1020.

The top layer 1046 and the bottom layer 1044 of the overmolding of the elastomeric material each form an outer surface of the needle guard assembly 1014. The overmolding of elastomeric material additionally forms a first intermediate layer 1024 between the first composite guard 1016 and the second composite guard 1018, and forms a second intermediate layer 1025 between the second composite guard 1018 and the third composite guard 1020.

The overmolding of elastomeric material may encapsulate the frame 1078. The frame 1078 may remain bonded to the composite guards 1016, 1018, 1020, such that when the needle guard assembly 1014 is implanted for use within a patient's body, the frame 1078 retains the relative positions of the composite guards 1016, 1018, 1020. In an embodiment in which the needle guard assembly 1014 is shaped to contour to the shape of a tube, for example a tube 502 shown in FIG. 23, the frame 1078 may be made sufficiently flexible to allow the needle guard assembly 1014 to wrap around the tube 502. In one embodiment, the frame 1078 may be removed from the assembly 1014 before the encapsulating layer is formed around the composite guards 1016, 1018, 1020. In this embodiment, the bonding of the encapsulating layer retains the relative positions of the composite guards 1016, 1018, 1020. The elastomeric material may comprise silicone, or the equivalent.

The needle guard assembly 1014 may be utilized as a needle guard, to protect a tubing from puncture, in the manner discussed in relation to FIGS. 20-25. The needle guard assembly 1014 may be utilized in combination with a clip 652, as shown in relation to FIGS. 26-28, or may be shaped to include a flanged portion, for example the flanged portion 770, shown in FIGS. 29-30. In one embodiment, the needle guard assembly 1014 may be utilized to protect an implantable device 310, for example the implantable device 310 shown and discussed in relation to FIGS. 11-12.

The puncture resistant members 1030, 1036, 1050 shown in FIGS. 33-38 may be made of a similar puncture resistant material as the puncture resistant members 430, 436, 530, 536, 550 discussed in relation to FIGS. 20-25. Namely, the members 1030, 1036, 1050 are made of a suitable puncture resistant material, such as an epoxy, acrylic materials, hot-melt adhesives, thermoplastics, polymer, rubber, ceramic or metal, or suitable combination or alloy thereof. For some applications, suitable materials include polyethylene (PE), polypropylene (PP), polyurethane (PU), polyethylene terephthalate (PET), polycarbonate (PC), polyisoprene (PI), thermoplastic urethanes and thermoplastic polyurethanes (TPU), high durometer silicones, acrylonitrile butadiene styrene (ABS) etc. In some embodiments, the members 1030, 1036, 1050 may be made from a UV-curable epoxy. In some embodiments, the members 1030, 1036, 1050 are made of material such as acetal, nylon, polycarbonate, and combinations thereof. In some embodiments, the members 1030, 1036, 1050 are made of a metal, for example, stainless steel, aluminum, titanium, or other metal.

The flexible substrates 1032, 1034, 1052 shown in FIGS. 33-38 may be made of a similar flexible material as the flexible substrates 432, 434, 532, 534, 552 discussed in relation to FIGS. 20-25. Namely, the flexible substrates 1032, 1034, 1052 may comprise a mesh, film, fabric, elastomer, or other suitable material. The flexible substrates 1032, 1034, 1052 may be made from thin polyimide, polyester, or other biocompatible film with appropriate thickness. The flexible substrates 1032, 1034, 1052 may be made from silicone, polyurethane, or other foam materials. The flexible substrates 1032, 1034, 1052 may be made from woven or non-woven mesh materials such as Nylon or Polyester. In one embodiment, the flexible substrates 1032, 1034, 1052 may be made from Kapton film. In one embodiment, the flexible substrates 1032, 1034, 1052 may be made from a polymer foam or plastic film.

The intermediate layers 1024, 1025 shown in FIGS. 37-38 may be made of a similar material as the intermediate layers 424, 524, 525 discussed in relation to FIGS. 20-25. Namely, the intermediate layers 1024, 1025 may be a polymer, for example, an elastomeric material, such as an elastomeric polymer, for example, a silicone elastomer, for example, a low durometer silicone rubber. The intermediate layers 1024, 1025 may comprise a soft, tacky layer of elastomeric material, generally comprised of silicone. The top layer 1046 and the bottom layer 1044 shown in FIGS. 37 and 38 may be made of similar materials discussed above for the intermediate layers 1024, 1025.

All materials used to form the needle guard assembly 1014 may be biocompatibility rated at USP Class VI. In addition, the encapsulating layer of an elastomeric material, forming a top layer 1046 and a bottom layer 1044 may be a grade of silicone designed for long term implantation (e.g., 5 or more years of implantation).

Unless otherwise indicated, all numbers expressing quantities of ingredients, volumes of fluids, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An implantable medical device for the treatment of obesity comprising:
an access port configured to attach to body tissue;
a tube having a first end and a second end, the first end coupled to the access port; and
a needle guard assembly covering the first end of the tube, the needle guard assembly including:
a first composite guard and a second composite guard, each composite guard including an arrangement of puncture resistant members and a flexible substrate having a first side on which the puncture resistant members are positioned,
the second composite guard covering the first composite guard, and
the first composite guard and the second composite guard being positioned such that the arrangement of puncture resistant members of the second composite guard is misaligned with the arrangement of puncture resistant members of the first composite guard.

2. The implantable device of claim 1 wherein the needle guard assembly is a sleeve that entirely encircles an outer surface of the first end of the tube.

3. The implantable device of claim 1 further comprising:
a first intermediate layer made of an elastomeric material, and positioned between and connecting the first composite guard with the second composite guard;
a bottom layer made of an elastomeric material, the first composite guard being positioned between the bottom layer and the first intermediate layer;
a third composite guard including an arrangement of puncture resistant members and a flexible substrate having a first side on which the puncture resistant members of the third composite guard are positioned, the arrangement of puncture resistant members of the third composite guard being misaligned with the arrangement of puncture resistant members of at least one of the second composite guard or the first composite guard;
a second intermediate layer made of an elastomeric material, and positioned between and connecting the second composite guard and the third composite guard; and
a top layer made of an elastomeric material, the top layer forming an outer surface of the needle guard assembly and being positioned such that the third composite guard is between the top layer and the second intermediate layer.

4. The implantable device of claim 3 wherein the bottom layer, the first intermediate layer, the second intermediate layer, and the top layer, are each made of implantable grade silicone.

5. The implantable device of claim 1 wherein each puncture resistant member of the first composite guard directly contacts an adjacent puncture resistant member of the first composite guard.

6. The implantable device of claim 1 wherein a space is positioned between each puncture resistant member of the first composite guard and an adjacent puncture resistant member of the first composite guard, and
a space is positioned between each puncture resistant member of the second composite guard and an adjacent puncture resistant member of the second composite guard.

7. The implantable device of claim 6 wherein the arrangement of puncture resistant members of the second composite guard is misaligned with the arrangement of puncture resistant members of the first composite guard, such that the puncture resistant members of the second composite guard cover each space positioned between each puncture resistant member of the first composite guard and the adjacent puncture resistant member of the first composite guard.

8. The implantable device of claim 6 wherein each space positioned between each puncture resistant member of the first composite guard and the adjacent puncture resistant member of the first composite guard, and each space positioned between each puncture resistant member of the second composite guard and the adjacent puncture resistant member of the second composite guard, has a size of between about 0.1 millimeter and about 1.0 millimeter.

9. The implantable device of claim 1 wherein each of the puncture resistant members of the first composite guard, and each of the puncture resistant members of the second composite guard, are made from a material selected from a group consisting of epoxy, acrylic, hot-melt adhesive, thermoplastic, polymer, rubber, ceramic, metal, and combinations thereof.

10. The implantable device of claim 1 wherein the flexible substrate of the first composite guard, and the flexible substrate of the second composite guard, are each made from a material selected from a group consisting of a mesh, a film, a fabric, an elastomer, and combinations thereof.

11. The implantable device of claim 1 wherein the puncture resistant members of the first composite guard, and the puncture resistant members of the second composite guard, each have a shape selected from a group consisting of a dome shape, a planar shape, and combinations thereof.

12. The implantable device of claim 1 wherein each of the puncture resistant members of the first composite guard, and each of the puncture resistant members of the second composite guard, has a thickness of between about 0.1 millimeter and about 1.0 millimeter.

13. The implantable device of claim 1 wherein each of the puncture resistant members of the first composite guard, and each of the puncture resistant members of the second composite guard, has a diameter of between about 0.5 millimeter and about 2.0 millimeters.

14. The implantable device of claim 1 further comprising a clip covering the needle guard assembly and securing the needle guard assembly to the first end of the tube.

15. The implantable device of claim 14 wherein the clip is secured to the access port.

16. The implantable device of claim 1 wherein the needle guard assembly is secured to an outer surface of the access port.

17. The implantable device of claim 1 wherein the first composite guard and the second composite guard each include registration holes, and
the needle guard assembly further comprises a frame extending through the registration holes of the first composite guard and the second composite guard to retain the arrangement of puncture resistant members of the second composite guard in a misaligned position relative to the arrangement of puncture resistant members of the first composite guard.

18. A needle guard assembly for protecting an implantable tube coupled to an implantable access port, the needle guard assembly comprising:
a first composite guard including an arrangement of puncture resistant members and a flexible substrate having a first side on which the puncture resistant members of the first composite guard are positioned;
a second composite guard including an arrangement of puncture resistant members and a flexible substrate having a first side on which the puncture resistant members of the second composite guard are positioned; and
a layer of elastomeric material forming an outer surface of the needle guard assembly,
the first composite guard, the second composite guard, and the layer of elastomeric material being layered relative to each other, and the first composite guard and the second composite guard being layered such that the arrangement of puncture resistant members of the second composite guard is misaligned with the arrangement of puncture resistant members of the first composite guard.

19. The needle guard assembly of claim 18 wherein the first composite guard and the second composite guard each include registration holes, and
the needle guard assembly further comprises a frame extending through the registration holes of the first composite guard and the second composite guard to retain the arrangement of puncture resistant members of the second composite guard in a misaligned position relative to the arrangement of puncture resistant members of the first composite guard.

20. The needle guard assembly of claim 18 wherein the layer of elastomeric material is part of an overmolding of the elastomeric material that encompasses the first composite guard and the second composite guard.

21. The needle guard assembly of claim 18 wherein the elastomeric material is implantable grade silicone.

22. The implantable device of claim 18 wherein each of the puncture resistant members of the first composite guard, and each of the puncture resistant members of the second composite guard, are made from a material selected from a group consisting of epoxy, acrylic, hot-melt adhesive, thermoplastic, polymer, rubber, ceramic, metal, and combinations thereof.

23. The implantable device of claim 18 wherein the flexible substrate of the first composite guard, and the flexible substrate of the second composite guard, are each made from a material selected from a group consisting of a mesh, a film, a fabric, an elastomer, and combinations thereof.

24. A gastric banding system for the treatment of obesity comprising:
a gastric band configured to form a loop around a portion of a patient's stomach to form a stoma;
an access port configured to attach to body tissue;
a tube having a first end and a second end, the first end coupled to the access port, and the second end coupled to the gastric band; and
a needle guard assembly covering the first end of the tube, the needle guard assembly including:
a first composite guard and a second composite guard, each composite guard including an arrangement of puncture resistant members and a flexible substrate having a first side on which the puncture resistant members are positioned,
the second composite guard covering the first composite guard, and
the first composite guard and the second composite guard being positioned such that the arrangement of puncture resistant members of the second composite guard is misaligned with the arrangement of puncture resistant members of the first composite guard.
